(19) United States
(12) Patent Application Publication
(10) Pub. No.: US 2021/0223233 A1
(43) Pub. Date: Jul. 22, 2021

(54) Mitochondrial Conductance Inhibitors and Methods of Use Thereof

(71) Applicant: The Regents of the University of California, Oakland, CA (US)

(72) Inventors: Yuriy Kirichok, Lafayette, CA (US); Francesca Fieni, San Francisco, CA (US)

(73) Assignee: The Regents of the University of California, Oakland, CA (US)

(21) Appl. No.: 15/733,874

(22) PCT Filed: May 21, 2019
(86) PCT No.: PCT/US2019/035081

§ 371 Date: Nov. 30, 2020

Related U.S. Application Data
(60) Provisional application No. 62/679,652, filed on Jun. 1, 2018.

(51) Int. Cl. G01N 33/50; A61K 31/155; A61K 31/4965; G01N 15/10

(52) U.S. Cl. CPC G01N 33/5076; A61K 31/155; G01N 2015/0038; G01N 15/1031; A61K 31/4965

(57) ABSTRACT

Disclosed herein is a newly discovered mitochondrial inner membrane nonspecific monovalent cation conductance. Methods of measuring the conductance are provided, including by patch clamp electrophysiology techniques, as well as methods of identifying inhibitors of the conductance. Inhibitors of the conductance are provided, as well as methods of using such inhibitors in the treatment of disease.

### CROSS-REFERENCE TO RELATED APPLICATIONS

This application claims the benefit of priority to International Patent Application Number PCT/US2019/035081, entitled “Mitochondrial Conductance Inhibitors and Methods of Use Thereof” filed May 31, 2019, which claims the benefit of priority to U.S. Provisional Patent Application Ser. No. 62/679,652, entitled “Mitochondrial Conductance Inhibitors and Methods of Use Thereof” filed on Jun. 1, 2018, each of which is incorporated by reference herein in its entirety.

### STATEMENT REGARDING FEDERALLY SPONSORED RESEARCH OR DEVELOPMENT

This invention was made with government support under grant nos. R01 GM107710 and OD004656 awarded by the National Institutes of Health. The government has certain rights in the invention.

### BACKGROUND OF THE INVENTION

The present disclosure relates to the discovery of a previously unknown nonselective monovalent cation conductance in the inner mitochondrial membrane. The methods and associated inventions disclosed herein are directed to the measurement of the conductance, the identification of inhibitors the conductance, inhibitors of the conductance, and methods of treatment of diseases associated mitochondrial function or state.

Metformin is a compound that is used to treat diabetes and has been shown to have several therapeutic effects, including decreasing glucose concentrations, ameliorating metabolic syndrome, and reducing the insulin dose requirement. Furthermore, Metformin has also been implicated as a potential cancer therapeutic, with studies demonstrating that it has a direct antitumor effect, which may depress tumor proliferation and induce the apoptosis, autophagy and cell cycle arrest of tumor cells, for example as described in Zi et al., Metformin and cancer: An existing drug for cancer prevention and therapy, Onco Letters, 2018 January; 15(1): 683-690.

Metformin has also been shown to improve cardiac function and has been proposed in the treatment of ischemic conditions, for example, reducing the severity and stroke symptoms and accelerating recovery and functional output stroke victims, for example, as described in Abassi et al., Impact of metformin on the severity and outcomes of ischemic stroke, Int J Basic Clin Pharm (2018) 7: 14. Furthermore metformin has protective and preventative effects on the development of heart disease, for example, as described in Nesti and Natali, Metformin effects on the heart and the cardiovascular system: A review of experimental and clinical data, Nutr. Metab. Cardiovasc Dis 2017 August; 27(8):657-669.

Various studies indicate that metformin has effects on mitochondrial function, for example, as described in Andrzejewski et al., Metformin directly acts on mitochondria to alter cellular bioenergetics, Cancer & Metabolism 2014, 2:12 and Boukalova et al., Mitochondrial Targeting of Metformin Enhances Its Activity against Pancreatic Cancer, 2016 December; 15(12):2875-2886. However, the specific mitochondrial activities by which metformin exerts its therapeutic effects are currently unknown.

Despite the great utility of metformin, large doses are often required to induce therapeutic effects.

Accordingly, there is a need in the art for an improved understanding of the mode of action by which metformin exerts therapeutic effects.

Additionally, there is a need in the art for drugs that share the therapeutic mode of action by which metformin works while having improved potency and optimized drug properties.

### SUMMARY OF THE INVENTION

The inventors of the present disclosure have determined that metformin acts by the inhibition of a previously unknown conductance in the mitochondrial inner membrane. Specifically, the conductance, newly disclosed herein, is a nonselective monovalent cation conductance that appears to act independently of known ion channels and carriers in the mitochondrial inner membrane. The newly discovered conductance will be referred to herein as the Nonselective Monovalent Cation Mitochondrial Inner Membrane Conductance, or “NMCC” for short.

Furthermore, the inventors of the present disclosure have discovered novel electrophysiology measurement techniques for the efficient measurement of the NMCC conductance.

Additionally, the inventors of the present disclosure have discovered inhibitors of the NMCC which may be used in the treatment of various diseases and conditions, for example, diseases and conditions that are treatable by metformin.

Accordingly, in a first aspect, the scope of the invention encompasses novel methods and experimental systems for performing electrophysiology measurements on the inner mitochondrial membrane to measure the NMCC.

In a next aspect, the scope of the invention encompasses novel methods and experimental systems for the efficient identification, characterization, and optimization of compositions that modulate the NMCC.

In one aspect, the scope of the invention encompasses the novel inhibitors of the NMCC.

In one aspect, the scope of the invention encompasses the novel use of previously known compositions as inhibitors of the NMCC.

In another aspect, the scope of the invention encompasses methods of modulating mitochondrial function or state by administration of an NMCC inhibitor.

In another aspect, the scope of the invention encompasses methods of treating mitochondrial-associated conditions by administration of an NMCC inhibitor.

In another aspect, the scope of the invention encompasses methods of treating diabetes and related conditions by administration of an NMCC inhibitor.

In another aspect, the scope of the invention encompasses methods of treating cancer by administration of an NMCC inhibitor.

In another aspect, the scope of the invention encompasses methods of treating cardiac and ischemic conditions and other cardiac conditions by administration of an NMCC inhibitor.

### BRIEF DESCRIPTION OF THE DRAWINGS

FIGS. 1A-1C depict an example of a mitochondrial patch-clamp technique. FIG. 1A shows a diagram of the preparation of a mitoplast from a mitochondrion. The outer mitochondrial membrane (OMNI) is ruptured and the inner mitochondrial membrane (IMM) is released to form the mitoplast. When the IMM is further released from the OMM, an 8-shaped vesicle is formed. The remnants of the OMM remain attached to the IMM in the initial mitoplast. FIG. 1B (left) shows a diagram of the mitoplast-attached configuration wherein a glass pipette recording electrode is used to form a giga-ohm seal with the IMM forming an 8-shaped mitoplast. FIG. 1B (right) shows a diagram of the mitoplast configuration which is formed when the IMM patch under the pipette recording electrode is ruptured by high-amplitude voltage pulses, and the IMM is nearly completely released from the OMM. After the IMM is ruptured, the mitoplast acquires a more rounded shape. FIG. 1C (top) shows a diagram of a whole-mitoplast patch-clamp recording of currents, such as monovalent cation currents, across a primarily intact IMM. FIG. 1C (bottom) shows an exemplary voltage protocol used to induce current. All voltages indicated correspond to the mitochondrial matrix in respect to the cytosol.

FIGS. 2A-2B depict a voltage-step protocol (FIG. 2A) and resulting sodium conductances from whole-mitoplast patch-clamp recordings (FIG. 2B) derived from a mouse heart mitochondrion.

FIGS. 3A-3B show exemplary recordings of Li⁺, K⁺, and Na⁺ conductances from whole-mitoplast patch-clamps (FIG. 3A) and the quantified current amplitudes from whole-mitoplast patch-clamp recordings of Li⁺, K⁺, and Na⁺ conductances (FIG. 3B).

FIGS. 4A-4E depict a voltage-step protocol (FIG. 4A); resulting whole-mitoplast recordings at pH 7.0 (FIG. 4B), pH7.5 (FIG. 4C), and pH 8.0 (FIG. 4D); and a graph summarizing the resulting sodium conductances from whole-mitoplast patch-clamp recordings recorded at pH 7.0, 7.5, and 8.0 (FIG. 4E).

FIGS. 5A-5E show exemplary recordings of K⁺ conductances from whole-mitoplast patch-clamps from heart (FIG. 5A), skeletal muscle (FIG. 5B), liver (FIG. 5C), kidney (FIG. 5D), and brown adipose UCP1−/− (FIG. 5E) mouse tissue.

FIGS. 6A-6B show exemplary recordings of K⁺ conductances from whole-mitoplast patch-clamps from drosophila (FIG. 6A) and C. elegans (FIG. 6B).

FIGS. 7A-7B show exemplary recordings of K⁺ conductance from whole-mitoplast patch-clamps in which the mitoplasts were not heated (FIG. 7A) or were heated (FIG. 7B). FIG. 7C depicts the current amplitudes quantified from whole-mitoplast patch-clamp recordings performed with heated and non-heated mitoplasts. FIG. 7D shows exemplary recordings of Na⁺ conductance from whole-mitoplast patch-clamps in the presence and absence of a high (mM) concentration of Ca²⁺.

FIGS. 8A-8C show recordings of exemplary monovalent cation conductances from whole-mitoplast patch-clamps in the presence or absence of ATP synthase inhibitors dicyclohexylcarbodiimide (DCCD) (FIG. 8A), oligomycin (FIG. 8B) or adenine nucleotide translocator inhibitor carboxyatractyloside (CATR) (FIG. 8C).

FIGS. 9A-9C show exemplary recordings of monovalent cation conductances from whole-mitoplast patch-clamps in the presence or absence of ATP-sensitive potassium channel (K_{ATP}) inhibitors arachidonic acid (AA) (FIG. 9A) and benzamil (FIG. 9B) or renal outer medullary potassium channel (ROMK) inhibitor Tertiapin Q (TQ) (FIG. 9C).

FIGS. 10A-10B show exemplary recordings of Na⁺ (FIG. 10A) or K⁺ (FIG. 10B) conductances from whole-mitoplast patch-clamps in the presence or absence of 5-(N-ethyl-N-isopropyl)amiloride (EIPA).

FIGS. 11A-11B show exemplary recordings of Na⁺ (FIG. 11A) or K⁺ (FIG. 11B) conductances from whole-mitoplast patch-clamps in the presence or absence of quinine.

FIG. 12 shows exemplary recordings of monovalent cation conductances from whole-mitoplast patch-clamps in the presence or absence of Complex IV inhibitor potassium cyanide (KCN).

FIGS. 13A-13C show exemplary recordings of monovalent cation conductances from whole-mitoplast patch-clamps in the presence or absence of Complex III inhibitors antimycin (FIG. 13A), stigmatellin (FIG. 13B), and myxothiazol (FIG. 13C).

FIG. 14 shows exemplary recordings of monovalent cation conductances from whole-mitoplast patch-clamps in the presence or absence of Complex II inhibitor atpenin.

FIGS. 15A-15C show exemplary recordings of monovalent cation conductances from whole-mitoplast patch-clamps in the presence or absence of rotenone (FIG. 15A), fenazaquin (FIG. 15B), and piericidin (FIG. 15C).

FIGS. 16A-16D show exemplary recordings of monovalent cation conductances from whole-mitoplast patch-clamps in the presence or absence of 1-methyl-4-phenylpyridinium (MPP+) (FIG. 16A), bullatacin (FIG. 16B), rolliniastatin (FIG. 16C), and fenpiroximate (FIG. 16D).

FIGS. 17A-17C show exemplary recordings of monovalent cation conductances from whole-mitoplast patch-clamps in the presence or absence of metformin (FIG. 17A), proguanil (FIG. 17B), and phenformin (FIG. 17C). FIG. 17D depicts the current amplitudes quantified from whole-mitoplast patch-clamp recordings performed as described for FIGS. 17A-17C.

FIGS. 18A-18C show exemplary recordings of monovalent cation conductances from whole-mitoplast patch-clamps in the presence or absence of AA6 (FIG. 18A), 5o (FIG. 18B), and 5m (FIG. 18C). FIG. 18D depicts the current amplitudes quantified from whole-mitoplast patch-clamp recordings performed as described for FIG. 18A-18C.

### DETAILED DESCRIPTION OF THE INVENTION

The various inventions disclosed herein are directed to both the measurement and the modulation of the nonselective monovalent cation inner mitochondrial membrane conductance referred to herein as the NMCC. The various elements of the inventions are described next.

### Definitions and Conventions

As used herein, the singular form “a,” “an,” and “the” includes plural references unless indicated otherwise.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that embodiments, aspects and variations described herein also include “consisting” and/or “consisting essentially of” embodiments, aspects and variations.

The term “effective amount” used herein refers to an amount of a compound or composition sufficient to treat a specified disorder, condition or disease, such as ameliorate, palliate, lessen, and/or delay one or more of its symptoms. In some embodiments, an effective amount means an amount of a compound or composition sufficient to induce a measurable biological effect or therapeutic effect. For example, in reference to a cancer, an effective amount comprises an amount sufficient to cause the number of cancer cells present in a subject to decrease in number and/or size and/or to slow the growth rate of the cancer cells. In some embodiments, an effective amount is an amount sufficient to prevent or delay recurrence of the disease. For example, in the case of cancer, the effective amount of the compound or composition may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer.

As used herein, a “mitoplast” is a vesicle derived from a mitochondrion with an outer mitochondrial membrane (OMNI) that has been at least partially or entirely removed and comprises an inner mitochondrial membrane (IMM) that remains primarily intact. A primarily intact inner mitochondrial membrane can include one or more perforations or ruptures to allow access to an inner compartment of the mitoplast by a probe, pipette, electrode, or other instrument.

As used herein, “whole-mitoplast” recording is analogous to a whole-cell recording applied to a mitoplast. Whole-mitoplast recording, as with whole-cell recording, requires rupturing the patch of membrane associated with the recording electrode, pipette, or other instrument to allow access to a transverse side of the membrane.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients, for example, have are those that meet the required standards of toxicological and manufacturing testing and/or are included on the Inactive Ingredient Guide prepared by the U.S. Food and Drug administration.

The term “subject” refers to a mammal and includes, but is not limited to, human, bovine, horse, feline, canine, rodent, or primate. In one embodiment, the subject is a human.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. For example, in reference to a cancer, the number of cancer cells present in a subject may decrease in number and/or size and/or the growth rate of the cancer cells may slow. In some embodiments, treatment may prevent or delay recurrence of the disease. In the case of cancer, the treatment may: (i) reduce the number of cancer cells; (ii) inhibit, retard, slow to some extent and preferably stop cancer cell proliferation; (iii) prevent or delay occurrence and/or recurrence of the cancer; and/or (iv) relieve to some extent one or more of the symptoms associated with the cancer. The methods of the invention contemplate any one or more of these aspects of treatment.

Unless defined otherwise, all terms of art, notations and other technical and scientific terms or terminology used herein are intended to have the same meaning as is commonly understood by one of ordinary skill in the art to which the claimed subject matter pertains.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### II. Methods of Measuring the Inner Mitochondrial Membrane Nonspecific Monovalent Cation Conductance

The section headings used herein are for organizational purposes only and are not to be construed as limiting the subject matter described. The scope of the invention encompasses methods of measuring and methods of inhibiting the NMCC. The NMCC may be defined in various ways. In one embodiment, the NMCC encompasses the conductance of monovalent cations through the inner mitochondrial membrane (IMM). In one embodiment, the NMCC is a nonspecific conductance for cations. In one embodiment, the NCMM is a monovalent cation conductance that can be inhibited by metformin. In one embodiment, the NCMM is a monovalent cation conductance that can be inhibited by 30 mM metformin. In one aspect, the NMCC is the conductance of potassium, sodium, and/or lithium through the IMM. In one aspect, the NMCC is a conductance of monovalent cations between the mitochondrial matrix and the intermembrane space. In one embodiment, the NMCC is a conductance between the mitochondrial matrix and the cytosol. In a primary embodiment, the NMCC comprises the conductance of monovalent cations through the inner mitochondrial membrane (IMM), wherein the conductance is nonspecific for monovalent cations and can be inhibited by metformin, for example, 30 mM metformin.

In one aspect, the scope of the invention encompasses systems and methods for measuring the NMCC. The measurement of the NMCC may be applied in various useful contexts, for example, studying the nature of the conductance and in the identification, characterization, and optimization of inhibitor and other modulators of the conductance.

The scope of the invention encompasses any method of qualitatively or quantitatively measuring the flow of monovalent cations through the inner mitochondrial membrane by the NMCC. Exemplary methods for measuring the NMCC include: using monovalent cation imaging, such as by the use of fluorescent Na⁺, K⁺, Li⁺, or Cs⁺ imaging; swelling assays; radio-labeling assays using a radioisotope of a monovalent cation; or patch-clamp recording, such as the patch-clamp recording methods described herein.

In a primary embodiment, the methods of the invention are directed to patch-clamp recording methods, such as any of the patch-clamp recording methods described herein, as such techniques provide a direct and specific method of measuring mitochondrial conductances.

Measurement Methods. The general measurement method of the invention encompasses the steps of:
obtaining one or more mitochondria from a selected source;
treating the one or more mitochondria to make the IMM accessible, wherein the one or more;
mitochondria are present in a bath solution;
applying a patch-clamp recording electrode to the IMM, wherein the patch-clamp recording electrode comprises a matrix solution;
by the patch-clamp recording electrode and an associated reference electrode, applying an electrical stimulus to induce the flow of monovalent cations across the IMM by the NMCC; and
by the patch clamp electrode, obtaining measurements that indicate the magnitude of the cation current flowing across the IMM, wherein the MCU conductance of the IMM is inhibited.

Methods of deriving or isolating mitochondria from tissues or cells are known in the art. For example, tissues or cells can be homogenized and the resulting homogenate can be centrifuged and mitochondria recovered. The source of the mitochondria may be any species, including, for example, humans, other primates, other mammals, for example, including rodents, dogs, cats, cows, and horses, zebrafish, insects, for example, drosophila, nematodes, yeast, fungi, plants, bacteria, and archaea. The source of the mitochondria may be, for example, whole organisms, explanted tissues, biopsy material, or cultured cells. The mitochondria used in the performance of the measurement methods may comprise mitochondria from any part of the selected organism, including from organs, tissues, tumors, single celled organisms, and any other source, for example, pancreatic cells, including pancreatic beta cells, liver cells, kidney cells, cardiac cells, muscle cells, including smooth muscle and skeletal muscle, and other cell types. In some embodiments, the source of the mitochondria is a human subjects at risk of or suffering from a selected condition. In some embodiments, the source of the mitochondria is a test animal, including animal models of disease. In some embodiments, the mitochondria utilized in the measurement methods of the invention comprise one or more mutations which impair the MCU, as described below.

Mitoplasts and Other IMM Presentations. The methods of the invention are applied in the measurement of monovalent cations across the IMM. As used herein, the IMM or inner mitochondrial membrane means the inner membrane of the mitochondria which separates the mitochondrial matrix from the intermembrane space (between the outer and inner mitochondrial membranes), as known in the art. The methods of the invention require treating the mitochondria by a physical and/or chemical treatment to make the IMM accessible to a patch-clamp electrode. In some embodiments, the mitochondria are treated in bulk to create a plurality of mitoplasts or otherwise accessible IMMs. In an alternative embodiment, an individual mitochondrion are manipulated to create mitoplasts or otherwise accessible IMMs.

In some implementations, mitochondria are treated to convert them to mitoplasts, which provide a convenient and physiologically intact structure for conducting patch clamp measurements.

Mitoplasts can be derived from mitochondria by methods known in the art, for example, as described in Kirichok et al., The mitochondrial calcium uniporter is a highly selective ion channel, (2004) Nature 427: 360-364. Mitoplasts may be formed by at least partially releasing the inner mitochondrial membrane (IMM) from the outer mitochondrial membrane (OMNI), for example by rupturing the OMNI. The OMM may be ruptured, for example, by applying a French press to mitochondria or by applying a hypotonic swelling solution to the mitochondria. In some embodiments, perforating the membrane comprises applying a molecule that forms perforations in the membrane. Exemplary molecules include nigericin or valinomycin. Methods of forming perforations are well known in the art.

In some embodiments, rupturing of the membrane comprises applying high suction or one or more electrical pulses to the membrane. The amount of high suction or electrical pulse necessary to rupture a membrane can vary from membrane to membrane. One of skill in the art can readily detect when a membrane has been ruptured. In some embodiments, the rupturing forms holes that are permeable to ions and molecules as large as 20-50 kDa. In some embodiments, the one or more electrical pulses are about 100 mV to about 800 mV, such as about 200 mV to about 700 mV, 600 mV, 550 mV, or 500 mV. In some embodiments, the one or more electrical pulses are about 200 mV to about 500 mV.

Typically, whole-mitoplast and perforated-patch recording measures produce a larger signal as these are measurements of conductance or voltage for proteins or complexes along the entire membrane. Mitoplast-attached recording or excised patch recording can also be used if it is desired to measure conductance or voltage for a single protein or complex. Accordingly, in some embodiments, the method comprises rupturing the mitochondrial membrane. In some embodiments, the method comprises perforating the mitochondrial membrane. In some embodiments, the method comprises excising a portion of the mitochondrial membrane.

In some embodiments, mitochondria are treated to form submitochondrial particles or proteoliposomes. A proteoliposome may be formed, for example, by mixing a mitochondrial membrane with a lipid microsome, which allows the membranes to fuse together.

In some implementations, the mitochondria are treated to form vesicles or a layer that divides a matrix side (i.e., the side of the membrane that is associated with the mitochondrial matrix given the orientation of the mitochondrial proteins) and a bath side. As discussed herein, the outer surface of the membrane or mitoplast refers to the surface of the membrane or mitoplast that contacts the bath, and the inner surface of the membrane or mitoplast refers to the surface of the membrane or mitoplast that contacts the matrix (or synthetic matrix).

In an alternative implementation, the mitochondria are not treated to form mitoplasts or otherwise expose or excise IMM. In these embodiments, the IMM is accessed in intact mitochondria through the outer mitochondrial membrane.

Solutions. The mitoplasts, or otherwise configured IMM sources, are formed in, or subsequent to their formation, are transferred to, a solution that will be referred to herein as the “bath solution.” The bath solution may comprise any number of compositions, described below.

The mitoplasts, or otherwise configured IMMs, will, in a next step, be probed with an electrode comprising a matrix solution. The matrix solution may comprise any number of compositions, described below.

The patch-clamp techniques of the invention are enabled by the use of solutions which facilitate the induction and measurement of NMCC currents while removing confounding currents and influences that obscure the measurable current. Mitochondrial membranes described herein have an inner surface and an outer surface. The inner surface is the surface of the membrane that would be in contact with the mitochondrial matrix in an intact mitochondrion given the orientation of the mitochondrial proteins. Accordingly, a solution that is applied to or contacting the inner surface is referred to as a matrix solution and a solution that is applied to or contacting the outer surface is referred to as a bath solution. Compositions of the matrix solutions and bath solutions described herein refer to the composition prior to application of a voltage.

The methods of the invention employ one or more selected monovalent cations in the solutions in order to enhance measurable signals of the NMCC. In one implementation, the matrix solution comprises a monovalent cation. In one implementation, the bath solution comprises a monovalent cation. In one implementation, both the matrix solution and the bath solution comprise a monovalent cation.

The one or more selected monovalent cations may comprise any cation capable of transport across the IMM by the NMCC. In some embodiments, the selected monovalent ion is K⁺, Li⁺, Cs⁺, Na⁺, and/or Tl⁺. In some embodiments, the monovalent cation is K⁺ and/or Cs⁺. In some embodiments, the monovalent cation is K⁺ or Na⁺. In some embodiments, the monovalent cation is K⁺. In some embodiments, the matrix solution comprises K⁺ and Na⁺. In some embodiments, the monovalent cation is tetramethylammonium (TMA), tetraethylammonium (TEA), N-methyl-D-glucamine (NMDG+), TRIS+, and/or an elemental monovalent cation.

In some embodiments, the matrix solution comprises the same monovalent cation as the bath solution. In some embodiments, the matrix solution and the bath solution each comprise K⁺. In some embodiments, the matrix solution and the bath solution each comprise Na⁺. In some embodiments, the matrix solution and the bath solution each comprise Li⁺. In some embodiments, the matrix solution and the bath solution each comprise Cs⁺. In some embodiments, the matrix solution comprises a second monovalent cation, which can be present or absent in the bath solution. In some embodiments, the matrix solution comprises a higher concentration of a monovalent cation than the bath solution. In some embodiments, the matrix solution comprises a lower concentration of a monovalent cation than the bath solution. In some embodiments, the matrix solution comprises the same concentration of a monovalent cation as the bath solution.

In some embodiments, the matrix solution comprises different monovalent cation than the bath solution. In some embodiments, the matrix solution does not comprise an elemental monovalent cation. In some embodiments, the matrix solution comprises TMA, TEA, NMDG+, or TRIS+ and the bath solution does not comprise TMA, TEA, NMDG+, or TRIS+. In some embodiments, the matrix solution comprises TMA and the bath solution comprises K⁺, Li⁺, Cs⁺, Na⁺, or Tl⁺.

In some embodiments, the matrix solution and/or the bath solution comprises the monovalent cation at a concentration of greater than about 10 mM, such as greater than about 20 mM, 50 mM, 80 mM, 90 mM, 100 mM, or 110 mM. In some embodiments, the matrix solution and/or the bath solution comprises the monovalent cation at a concentration of less than about 300 mM, such as less than about 250 mM, 225 mM, 200 mM, 175 mM, 150 mM, or 125 mM. In some embodiments, the matrix solution and/or the bath solution comprises the monovalent cation at a concentration of about 10 mM to about 300 mM, such as about 50 mM to about 200 mM, 175 mM, 150 mM, 125 mM, or 110 mM; about 100 mM to about 175 mM; or about 100 mM to about 150 mM.

In some embodiments, the monovalent cations of the matrix solution and/or the bath solution are provided as the salt of a selected anion. In one implementation, the selected anion is gluconate. In other embodiments, the selected anion is glucuronate, nitrate, sulfate, glutamate, pyruvate, lactate, ethanesulfonate, or chloride salt of a monovalent cation. Measurements of the NCMM may be confounded or obscured by the coincidental or counterflow of anions, for example, monovalent ions, particularly chloride. Accordingly, in some implementations, the cations of the matrix or bath solution are provided in the form of gluconate salts, as gluconate ions, due to their charge and bulk, do not substantially cross the IMM and thus their use removes the confounding influence of anion conductance across the IMM of the selected cation.

In some implementations, it is desirable to substantially remove divalent cations from the matrix solution, bath solution, or both. In some embodiments, the matrix solution comprises a very low concentration of divalent cations, for example, concentrations of less than 1 mM, less than 100 micromolar, less than 10 micromolar, less than 1 micromolar, or the solution substantially does not comprise a divalent cation. In some embodiments, the bath solution comprises a very low concentration of divalent cations, for example, concentrations of less than less than 1 mM, less than 100 micromolar, less than 10 micromolar, less than 1 micromolar, or the solution substantially does not comprise a divalent cation. In some embodiments, the matrix solution comprises a divalent cation and the bath solution does not comprise a divalent cation. In some embodiments, the matrix solution does not comprise Mg²⁺. In some embodiments, the matrix solution does not comprise Ca²⁺. In some embodiments, the bath solution does not comprise Mg²⁺. In some embodiments, the bath solution does not comprise Ca²⁺. In some embodiments, the bath solution comprises a divalent cation and the matrix solution does not comprise a divalent cation. In some embodiments, the matrix solution comprises K⁺ or Cs⁺ and does not comprise Ca²⁺. In some embodiments, the bath solution comprises K⁺ or Cs⁺ and does not comprise Ca²⁺.

The removal of divalent cations from the matrix and/or bath solution may be achieved by the use of a chelator in the solution. In some embodiments, the bath solution comprises a chelator. In some embodiments, the matrix solution comprises a chelator. In some embodiments, the bath solution comprises a chelator. In some embodiments, both the matrix solution and the bath solution comprise a chelator. In some embodiments the chelator is (1,2-bis(o-aminophenoxy)ethane-N,N,N′,N′-tetraacetic acid (BAPTA), deferoxamine, diethylenetriaminepentaacetic acid (DTPA), 2,3-dimercapto-1-propanesulfonic acid (DMPS), dimercaptosuccinic acid (DMSA), polyaspartic acid ethylenediamine-N,N′-disuccinic acid (EDDS), ethylenediaminetetraacetic acid (EDTA), ethylene glycol-bis(β-aminoethyl ether)-N,N,N′,N′-tetraacetic acid) (EGTA), methylglycinediacetic acid, L-glutamic acid N,N-diacetic acid, tetrasodium salt (GLDA), iminodisuccinic acid (IDS), or lipoic acid (LA), or a mixture of any of the foregoing. In some embodiments, the chelator is EDTA or EGTA. In some embodiments, the matrix and/or bath solution comprises two chelators. In some embodiments, the two chelators are EDTA and EGTA.

In some embodiments, the matrix solution and/or the bath solution comprises a chelator, which may be a single compound (e.g., EDTA) or a combination of two or more compounds (e.g., EDTA and EGTA), at a concentration of greater than about 500 μM, such as greater than about 600 μM, 700 μM, 800 μM, or 900 μM. In some embodiments, the matrix solution and/or the bath solution comprises a chelator, which may be a single compound (e.g., EDTA) or a combination of two or more compounds (e.g., EDTA and EGTA), at a concentration of less than about 20 mM, such as less than about 15 mM, 10 mM, 8 mM, 7 mM, 6 mM, or 5 mM. In some embodiments, the matrix solution and/or the bath solution comprises the chelator, which may be a single compound (e.g., EDTA) or a combination of two or more compounds (e.g., EDTA and EGTA), at a concentration of about 500 μM to about 20 mM, such as about 750 μM to about 15 mM, 10 mM, 8 mM, 7 mM, 6 mM, or 5 mM; or about 1 mM to about 5 mM.

In some embodiments, the matrix solution comprises a buffer. In some embodiments, the bath solution comprises a buffer. In some embodiments, the matrix solution and the bath solution both comprise a buffer. In some embodiments, the buffer is acetamidoglycine, N-(2-Acetamido)-2-aminoethanesulfonic acid (ACES), acetic acid, N-(2-acetamido)iminodiacetic acid (ADA), aminomethyl propanol, (3-aminomethylphenyl)boronic acid (AMPB), N-(1,1-Dimethyl-2-hydroxyethyl)-3-amino-2-hydroxypropanesulfonic acid (AMPSO), N-Bis(2-hydroxyethyl)-2-aminoethanesulfonic acid (BES) bicine, bis-tris methane, borate, cacodylate, 4-(Cyclohexylamino)-1-butanesulfonic acid (CABS), N-cyclohexyl-3-aminopropanesulfonic acid (CAPS), N-Cyclohexyl-2-aminoethanesulfonic acid (CHES), Cholamine chloride hydrochloride, citric acid, 3-(N,N-Bis[2-hydroxyethyl]amino)-2-hydroxypropanesulfonic acid (DIPSO) glycinamide, glycylglycine, N-(2-Hydroxyethyl)piperazine-N′-(4-butanesulfonic acid) (HEPBS), 4-(2-hydroxyethyl)-1-piperazineethanesulfonic acid (HEPES), 3-[4-(2-Hydroxyethyl)piperazin-1-yl]propane-1-sulfonic acid (HEPPS), N-(2-Hydroxyethyl)-piperazine-N′-2-hydroxypropanesulfonic acid (HEPPSO) 2-(N-morpholino)ethanesulfonic acid (MES), 4-(N-Morpholino)butanesulfonic acid (MOBS), 3-(N-morpholino)propanesulfonic acid (MOPS), 2-Hydroxy-3-morpholinopropanesulfonic acid (MOPSO), phosphate buffer, piperazine-N,N′-bis(2-ethanesulfonic acid) (PIPES), piperazine-N,N′-bis(2-hydroxypropanesulfonic acid) (POPSO), [tris(hydroxymethyl)methylamino]propanesulfonic acid (TAPS), 3-[N-Tris(hydroxymethyl)methylamino]-2-hydroxypropanesulfonic acid (TAPSO), 2-[[1,3-dihydroxy-2-(hydroxymethyl)propan-2-yl]amino]ethanesulfonic acid (TES), tricine, triethanolamine, or tris. In some embodiments, the buffer is HEPES.

In some embodiments, the matrix solution and/or the bath solution comprises a buffer at a concentration of greater than about 1 mM, such as greater than about 2 mM, 5 mM, 8 mM, 10 mM, 15 mM, 20 mM, 25 mM, 30 mM, 40 mM, 50 mM, 75 mM, or 100 mM. In some embodiments, the matrix solution and/or the bath solution comprises a buffer at a concentration of less than about 300 mM, such as less than about 200 mM, 175 mM, 150 mM, 100 mM, 75 mM, 60 mM, 50 mM, 40 mM, or 30 mM. In some embodiments, the matrix solution and/or the bath solution comprises a buffer at a concentration of about 1 mM to about 300 mM, such as about 10 mM to about 200 mM, 170 mM, 150 mM, 125 mM, 100 mM, 75 mM, 60 mM, or 40 mM; about 100 mM to about 170 mM; or about 20 mM to about 60 mM.

In some embodiments, the matrix solution comprises a monovalent anion. In some embodiments, the bath solution comprises a monovalent anion. In some embodiments, both the matrix solution and the bath solution comprises a monovalent anion. In some embodiments, the monovalent anion is gluconate⁻or Cl⁻. In some embodiments, the matrix solution and/or the bath solution comprises the monovalent anion at a concentration of greater than about 10 mM, such as greater than about 20 mM, 50 mM, 80 mM, 90 mM, 100 mM, or 110 mM. In some embodiments, the matrix solution and/or the bath solution comprises the monovalent anion at a concentration of less than about 300 mM, such as less than about 250 mM, 225 mM, 200 mM, 175 mM, 150 mM, or 125 mM. In some embodiments, the matrix solution and/or the bath solution comprises the monovalent anion at a concentration of about 10 mM to about 300 mM, such as about 50 mM to about 200 mM, 175 mM, 150 mM, 125 mM, or 110 mM; about 100 mM to about 175 mM; or about 100 mM to about 150 mM.

In some embodiments, the matrix solution comprises a divalent cation. In some embodiments, the bath solution comprises a divalent cation. In some embodiments, the matrix solution and the bath solution comprise a divalent cation. In some embodiments, the divalent cation is Mg²⁺ or Ca²⁺. In some embodiments, the matrix solution and/or the bath solution comprises the divalent cation at a concentration of greater than about 50 nM, such as greater than about 75 nM, 90 nM, or 100 nM. In some embodiments, the matrix solution and/or the bath solution comprises the divalent cation at a concentration of less than about 1 mM, such as less than about 500 μM, 100 μM, 50 μM, 10 μM, 5 μM, 1 μM, 500 nM, 400 nM, or 300 nM. In some embodiments, the matrix solution or the bath solution comprises the monovalent anion at a concentration of about 50 nM to about 500 such as about 100 nM to about 100 μM, 50 μM, 10 μM, 5 μM, 1 μM, 500 nM, 400 nM, or 300 nM.

In some embodiments, the matrix solution comprises an antioxidant. In some embodiments, the bath solution comprises an antioxidant. In some embodiments, the matrix solution and the bath solution comprise an antioxidant. In some embodiments, the antioxidant is glutathione.

In some embodiments, the matrix solution and/or the bath solution comprises the antioxidant at a concentration of greater than about 500 such as greater than about 750 μM, 1 mM, 5 mM, or 10 mM. In some embodiments, the matrix solution and/or the bath solution comprises an antioxidant at a concentration of less than about 50 mM, such as less than about 30 mM, 25 mM, 20 mM, 19 mM, 18 mM, or 17 mM. In some embodiments, the matrix solution and/or the bath solution comprises an antioxidant at a concentration of about 500 μM to about 50 mM, such as about 1 mM to about 30 mM, 25 mM, 20 mM, 19 mM, 18 mM, 17 mM; or about 10 mM to about 15 mM.

In some embodiments, the matrix solution, the bath solution, or both the matrix solution and the bath solution have an osmolarity of about 250 to about 500 mmol/kg, such as about 300 mmol/kg to about 500 mmol/kg, 475 mmol/kg, 450 mmol/kg, or 400 mmol/kg; about 330 mmol/kg to about 500 mmol/kg, 475 mmol/kg, 450 mmol/kg, or 400 mmol/kg, 375 mmol/kg, or 350 mmol/kg.

In some embodiments, the matrix solution comprises a sugar or sugar alcohol. In some embodiments, the bath solution comprises a sugar or sugar alcohol. In some embodiments, both the matrix and the bath solution comprise a sugar or sugar alcohol. In some embodiments, the sugar or sugar alcohol is mannitol, sucrose, glucose, galactose, lactose, maltose, or fructose. In some embodiments, the sugar is sucrose.

In some embodiments, the matrix solution has a concentration of the sugar or sugar alcohol that allows for an osmolarity of the solution of about 250 to about 500 mmol/kg, such as about 300 mmol/kg to about 500 mmol/kg, 475 mmol/kg, 450 mmol/kg, or 400 mmol/kg; about 330 to about 500 mmol/kg, 475 mmol/kg, 450 mmol/kg, or 400 mmol/kg. In some embodiments, the bath solution has a concentration of the sugar or sugar alcohol that allows for an osmolarity of the solution of about 250 to about 500 mmol/kg, such as about 330 mmol/kg to about 500 mmol/kg, 475 mmol/kg, 450 mmol/kg, 400 mmol/kg, 375 mmol/kg, or 350 mmol/kg.

In some embodiments, the matrix solution has a pH between 6.5 and 9. In some embodiments, the bath solution has a pH between 6.5 and 9. In some embodiments, the pH of the matrix and/or bath solution is about 7.0 to about 8.0, such as about 7.0 to about 7.8, 7.6, 7.5, 7.4, 7.3, or 7.2. In some embodiments, the pH of the matrix and/or bath solution is about 7.0, 7.2, 7.5, or 8.0. Methods of measuring pH are known in the art, for example using a pH detecting probe, litmus paper, or pH indicating compounds. Methods of adjusting the pH of a solution are also known in the art. Various compounds can be used to adjust the pH of a solution, such as NaOH, KOH, D-gluconic Acid, or TRIS base.

Modulation of MCU and Other IMM Currents. In order to efficiently measure the NMCC, in some implementations, the activity of the mitochondrial calcium uniporter (MCU) is inhibited or ablated. Inhibition of the MCU removes calcium fluxes through the IMM which may confound the measurement of the NMCC. Inhibition of the MCU may encompass any reduction in MCU conductance or calcium currents, for example, measured relative to non-inhibited MCU activity. For example, the reduction in MCU activity may be of at least 50%, at least 60%, at least 70%, at least 80%, at least 90%, at least 95%, at least 99% or total elimination of detectable MCU activity.

The inhibition of MCU activity may be achieved by various means. In a first implementation, MCU activity is achieved by the use of bath and/or matrix solutions the reduce MCU activity. In a first embodiment, MCU activity is inhibited by the removal of calcium from the matrix and/or bath solutions, for example, by reducing calcium concentrations to below 1 mM, below 100 micromolar, below 10 micromolar, below 1 micromolar, below 100 nanomolar, below 10 nanomolar, or less.

In a second implementation, MCU activity is reduced by removal of divalent cations with the measurement of K⁺ or Cs⁺ fluxes as the measure of the NMCC, as the MCU is not permeable to these monovalent cations in the absence of a divalent cation.

In another implementation, the bath and/or matrix solution comprises an inhibitor of the MCU, for example, one or more inhibitors selected from the group consisting of binuclear oxo-bridged ruthenium complexes such as ruthenium 360, nitrido-bridged ruthenium complexes, DS16570511, hexamine cobalt chloride, and other MCU inhibitors known in the art.

In another implementation, MCU activity is inhibited by the use of mitochondria comprising one or more mutations which reduce or eliminate MCU activity. In one embodiment, the mitochondria comprises a knocked out or knocked down MCU, for example, knockdown by siRNA techniques, as known in the art.

In addition to the MCU, other potentially confounding currents and/or background signals may be present that obscure or complicate NMCC measurement. These may be eliminated by various means. In some embodiments, a mitochondrion that does not express a fully functioning proton channel is utilized. In some embodiments, the mitochondrion comprises a mutant mitochondrial uncoupling protein. In some embodiments, the mitochondrion does not comprise a mitochondrial uncoupling protein.

Exemplary Solutions. Various formulations of the bath and matrix solutions may be used. In one embodiment, the bath solution and/or the matrix solution comprises:
a monovalent cation, for example, a gluconate salt of Na-gluconate, TMA-gluconate, K-gluconate, Li-gluconate, or Cs-gluconate;
a chelator which removes substantially all divalent cations from the solution; for example as chelator selected from the group consisting of (1,2-bis(o-aminophenoxy)ethane-N,N,N′,N′-tetraacetic acid (BAPTA), deferoxamine, diethylenetriaminepentaacetic acid (DTPA), 2,3-dimercapto-1-propanesulfonic acid (DMPS), dimercaptosuccinic acid (DMSA), polyaspartic acid ethylenediamine-N,N′-disuccinic acid (EDDS), ethylenediaminetetraacetic acid (EDTA), ethylene glycol-bis(β-aminoethyl ether)-N,N,N′,N′-tetraacetic acid) (EGTA), methylglycinediacetic acid, L-glutamic acid N,N-diacetic acid, tetrasodium salt (GLDA), iminodisuccinic acid (IDS), or lipoic acid (LA), or a mixture of any of the foregoing; and
a buffer, for example a buffer selected from the group consisting of ACES, ADA, AMPB, AMPSO, BES, CABS, CHES, DIPSO, HEPBS, HEPES, HEPPS, HEPPSO, MOBS, MOPS, MOPSO, PIPES, POPSO, TAPS, TAPSO, TES, tricine, triethanolamine, and tris;
a sugar or sugar alcohol, for example sucrose;
wherein the pH of the solution is between about 6.5 and 9, for example a pH of 7.0 to 7.5; and
wherein the osmolality of the solution is between about 250 to about 500 mmol/kg, for example, an osmolality about 330-450 mmol/kg.

An exemplary matrix solution is pH 7 to 7.5 and comprises one or more of: (i) 100 mM-150 mM Na-gluconate, TMA-gluconate, NaCl, K-gluconate, KCl, Li-gluconate, LiCl, Cs-gluconate, CsCl (ii) 1-5 mM EGTA, (iii) 1-5 mM EDTA, (iv) 20-100 mM HEPES, (v) an amount of sucrose that allows the solution to have an osmolarity of 330-450 mmol/kg, and (vi) 1 mM NaCl. In some embodiments, the matrix solution comprises any 1 of (i)-(iv), any 2 of (i)-(iv), any 3 of (i)-(iv), any 4 of (i)-(iv), any 5 of (i)-(iv), or all of (i)-(iv).

In another example, the matrix solution is pH 7 to 7.5 and comprises one or more of: (i) 100 mM-175 mM TMA, (ii) 1-5 mM EGTA, (iii) 10-150 mM HEPES, (iv) 0-15 mM glutathione, (v) 1-3 mM MgCl₂, NaCl, KCl, or TrisCl, (vi) an amount of sucrose that allows the solution to have an osmolarity of 330-500 mmol/kg, and (vi) 1 mM NaCl. In some embodiments, the matrix solution comprises any 1 of (i)-(iv), any 2 of (i)-(iv), any 3 of (i)-(iv), any 4 of (i)-(iv), any 5 of (i)-(iv), or all of (i)-(iv).

An exemplary bath solution is pH 7 to 7.5 and comprises one or more of: (i) 100 mM-150 mM Na-gluconate, NaCl, K-gluconate, KCl, Li-gluconate, LiCl, Cs-gluconate, CsCl; (ii) 1-5 mM EGTA; (iii) 1-5 mM EDTA; (iv) 10-100 mM HEPES; and (v) an amount of sucrose that allows the solution to have an osmolarity of 250-330 mmol/kg. In some embodiments, the bath solution comprises any 1 of (i)-(iv), any 2 of (i)-(iv), any 3 of (i)-(iv), any 4 of (i)-(iv), any 5 of (i)-(iv), or all of (i)-(iv).

Measurement of the MNCC. The measurement methods of the invention encompass the detection and/or quantification of a current in the IMM, for example, by patch-clamp techniques. A variety of patch-clamp techniques are known in the art. Patch-clamping typically requires a recording electrode connected to an amplifier and a reference or ground electrode.

Suitable recording electrodes are known in the art, such as pipette electrodes or planar electrodes. For patch-clamping techniques, recording electrodes should be capable of forming a gigaohm seal and conducting electricity. Pipette electrodes typically comprise a poorly conducting pipette or tube, such as a glass or fused quartz pipette, an ionic aqueous solution, such as any of the matrix solutions described herein, and a conducting material, such as a metallic wire. Planar electrodes typically comprise a planar, poorly conducting surface, an ionic aqueous solution, such as any of the matrix solutions described herein, and a conducting material, such as a metallic wire. Planar electrodes may be individual electrodes or part of a larger chip that allows for multiple patch-clamps to occur in parallel.

The electrodes of the patch clamp system will be in connection with electronic components for the application of voltages, amplifier readout, and signal processing and storage. Exemplary components include a potentiostat for precise application of voltages in selected sequences, readout components such as analog to digital converters and memory elements for recording of data, and computerized control elements.

In a primary application, the patch clamp technique will be a whole-mitoplast patch clamp recording, wherein the patch of membrane contacted by the recording electrode is ruptured. This allows for recording from the whole mitoplast, rather than just the portion of the membrane in contact with the membrane. Other patch-clamping techniques contemplated include mitoplast-attached recording, perforated-patch recording, or excised patch recording, such as inside-out or outside-out patch.

In a primary implementation, the mitoplast-patch clamp technique is a voltage clamp, wherein the voltage across the cell membrane is controlled and the resulting currents are recorded. In an alternative implementation, a current clamp technique is utilized, wherein the current passing across the membrane is controlled and the resulting changes in voltage are recorded.

In the methods of the invention, the recording electrode, for example, a pipette electrode, is brought into contact with the IMM membrane, such as the exposed IMM of a mitoplast. This procedure is performed by a user with the aid of suitable microscopy and imaging systems and staging systems for the fine manipulation of recording electrode movement. Upon contacting the IMM, a gigaohm seal, i.e. a seal with a resistance in the gigaohm range, is formed, for example by applying suction to the inner lumen of the pipette electrode. The reference ground electrode is also placed in the bath surrounding the mitoplast or otherwise accessible IMM.

Following contact with the recording electrode and formation of a seal, a voltage may be applied to the IMM by the electrodes. In some embodiments, the voltage applied is about −200 mV to about +200 mV, such as about −200 mV to about +175 mV, +150 mV, +125 mV, or +100 mV. In some embodiments, the voltage applied is about −180 mV to about 100 mV. In some embodiments, a single voltage is applied. In some embodiments, multiple voltages are applied. In some embodiments, the voltage applied is gradually increased or decreased over a period of time. In some embodiments, the voltage applied is quickly increased or decreased, such as increased or decrease for over a period of time that is less than 10 ms, such as less than 5 ms, 4 ms, 3 ms, 2 ms, or 1 ms. In some embodiments, the voltage applied is quickly increased or decreased and then gradually increased or decreased over a period of time. In some embodiments, the period of time is about 10 ms to about 1 h, such as about 30 min, 1 min, 30 s, 10 s, 1 s, 500 ms, or less. In some embodiments, the period of time about 500 ms to about 1 s.

Recording electrode output is recorded by the signal processing components in connection with the electrodes. Electrode output may be represented as a trace or graph depicting currents over time. Outputs may be processed by computer to calculate currents, including raw currents, corrected currents, or cumulative currents.

Heating Treatment. In some embodiments, the IMMs, for example, of mitoplasts, are heated prior to and/or during the measurement process. For example, the mitochondrial membrane can be heated to temperatures of greater than 30° C., for example, temperatures of between 30 and 40° C., for example temperatures of about 37° C., for a period of time. For example, the period of time may be between 5 and 30 minutes, for example, for about 10-15 minutes. Heating may be achieved by heating the bath solution in which the IMMs, e.g. mitoplasts, are present.

In an alternative to heating, mitoplasts are preserved in a cooled state for a period of time prior to measurement of the NMCC. For example, bath solutions may be cooled on ice or otherwise cooled, for example at temperatures of about 0-4° C. for a period of time prior to measurement. For example, a cooling period of 15-60 minutes may be employed, for example, about 30 minutes.

Identification, Characterization, and Optimization of NMCC Inhibitors. In one implementation, the scope of the invention encompasses the measurement of the NMCC wherein a test compound comprising an NMCC inhibitor or putative inhibitor is included in the matrix solution, bath solution, or both solutions. By comparison to control treatments lacking an inhibitor or putative inhibitor, the effects of the test compound can be quantified.

In some embodiments, the matrix solution comprises the test compound. In some embodiments, the bath solution comprises test compound. In some embodiments, the matrix solution and the bath solution comprise the test compound. In some embodiments, the matrix solution and/or the bath solution comprises the test compound at a concentration of greater than about 10 nM, such as greater than about 50 nM, 100 nM, 500 nM, 1 μM, 5 μM, 10 μM, 50 μM, 100 μM, 500 μM, or 1 mM. In some embodiments, the matrix solution and/or the bath solution comprises the test compound at a concentration of less than about 1 mM, such as less than about 500 μM, 100 μM, 50 μM, 10 μM, 5 μM, 1 μM, 500 nM, 100 nM, or 50 nM. In some embodiments, the matrix solution and/or the bath solution comprises the test compound at a concentration of about 1 nM to about 100 mM, such as about 10 nM to about 50 mM, 1 mM, 500 μM, 100 μM, 50 μM, 10 μM, 5 μM, 1 μM, 500 nM, 100 nM, or 50 nM; about 100 nM to about 10 μM; about 1 μM to about 100 μM; or about 100 μM to about 1 mM.

By comparison to control treatments lacking the test compound, the NMCC inhibitory properties of a selected composition of matter may be assessed. This enables characterization of the selected inhibitor or putative inhibitor of the NMCC, for example in the optimization of inhibitors by changing chemical substituents.

In one implementation, the NMCC measurement methods of the invention are utilized to screen putative NMCC inhibitors for inhibitory activity. In this method, the NMCC is measured in the presence and absence of a test compound comprising a putative inhibitor of the NMCC, wherein, if NMCC is reduced by the presence of the test compound, for example, by a selected threshold level of inhibition (e.g. at least 10% inhibition, at least 20% inhibition, at least 30% inhibition, at least 40% inhibition, at least 50% inhibition, at least 60% inhibition, at least 70% inhibition, at least 80% inhibition, or at least 90% inhibition), the test compound is deemed to be an inhibitor of the NMCC. By this method, any number of compounds may be screened and effective inhibitors of the NMCC may be identified.

In some embodiments, the screening method is repeated for more than one test compound. In some embodiments, the method is repeated for at least 2, at least 5, at least 10, at least 20, at least 50, at least 100, at least 500, or at least 1000, or more putative NMCC inhibitors or test compounds. In some embodiments, the contacting the mitochondrial membrane comprises contacting with two or more compounds simultaneously, such as three, four, or more compounds. In some embodiments, the compound is applied to an outer surface of the mitochondrial membrane. In some embodiments, the compound is applied to an inner surface of the mitochondrial membrane.

In some embodiments, the matrix solution comprises the putative NMCC inhibitor or test compound. In some embodiments, the bath solution comprises the putative NMCC inhibitor or test compound. In some embodiments, the matrix solution or the bath solution comprises the putative NMCC inhibitor or test compound at a concentration of greater than about 10 nM, such as greater than about 50 nM, 100 nM, 500 nM, 1 μM, 5 μM, 10 μM, 50 μM, 100 μM, 500 μM, or 1 mM.

In some embodiments, the putative NMCC inhibitor or test compound is an unknown compound. In some embodiments, the putative NMCC inhibitor or test compound is a small molecule compound or a polypeptide. In some embodiments, the polypeptide is an antibody or antigen binding fragment thereof. In some embodiments, the identifying may comprise analyzing the compound, such as determining the structure of the compound using techniques known in the art. Such techniques include, but are not limited to, mass spectrometry, nuclear magnetic resonance, infrared spectroscopy, etc. Also provided herein are NMCC inhibitors identified by any of the methods described herein.

### III. Inhibitors of the Inner Mitochondrial Membrane Nonselective Monovalent Cation Conductance and Formulations Thereof

The inventors of the present disclosure have advantageously identified compositions of matter that inhibit the NMCC. In some embodiments, the inhibitor of mitochondrial conductance inhibits in vivo NMCC, wherein the NMCC is an IMM conductance for monovalent cations and/or a monovalent cation conductance that can be inhibited by metformin, such as can be assessed via the methods detailed herein. In some embodiments, the inhibitor of NMCC inhibits in vitro mitochondrial conductance, wherein the NMCC is an IMM conductance for monovalent cations and/or a monovalent cation conductance that can be inhibited by metformin, such as can be assessed via the methods disclosed herein. In some embodiments, the NMCC is associated with the electron transport chain.

The inhibited conductance may encompass the inhibition of any monovalent cation across the IMM, including, for example, K⁺, Li⁺, Cs⁺, Na⁺, and Tl⁺. In some embodiments, the inhibited NMCC is inhibited K⁺, Li⁺, or Na⁺ conductance. In some embodiments, the inhibited NMCC is inhibited K⁺ or Na⁺ conductance. In some embodiments, the inhibited NMCC is inhibited K⁺ conductance.

In some embodiments, the inhibitor measurably inhibits NMCC at a concentration of about 10 μM or less, such as at a concentration of about 9 μM or less, about 7 μM or less, about 5 μM or less, about 3 μM or less, about 2 μM or less, about 1 μM or less, about 0.9 μM or less, about 0.8 μM or less, about 0.7 μM or less, about 0.6 μM or less, or about 0.5 μM or less.

In some embodiments, the inhibitor inhibits the NMCC in vitro at a concentration of about 10 μM or less, such as at a concentration of about 9 μM or less, about 7 μM or less, about 5 μM or less, about 3 μM or less, about 2 μM or less, about 1 μM or less, about 0.9 μM or less, about 0.8 μM or less, about 0.7 μM or less, about 0.6 μM or less, or about 0.5 μM or less.

In some embodiments, in vitro inhibition of the NMCC is verified by measuring the mitochondrial conductance for monovalent cations in the presence of and absence of the inhibitor.

In some embodiments, the inhibitor inhibits at least about 10%, such as at least about 15%, 20%, 25%, 30%, 35%, 40%, 45%, 50%, 55%, 60%, 65%, 70%, 75%, 80%, or 90% of the NMCC. In some embodiments, the inhibitor of inhibits at least about 10%, such as at least about 15%, 20%, 25%, 30%, 35%, 40%, 45%, 50%, 55%, 60%, 65%, 70%, 75%, 80%, or 90% of the NMCC in vitro. In some embodiments, the inhibitor of inhibits at least about 10%, such as at least about 15%, 20%, 25%, 30%, 35%, 40%, 45%, 50%, 55%, 60%, 65%, 70%, 75%, 80%, or 90% of the NMCC in vitro as determined by a patch-clamp recording. In some embodiments, the inhibitor inhibits at least about 10%, such as at least about 15%, 20%, 25%, 30%, 35%, 40%, 45%, 50%, 55%, 60%, 65%, 70%, 75%, 80%, or 90% of the NMCC in vitro as determined by a whole-mitoplast patch-clamp recording. In some embodiments, the inhibitor inhibits at least about 10%, such as at least about 15%, 20%, 25%, 30%, 35%, 40%, 45%, 50%, 55%, 60%, 65%, 70%, 75%, 80%, or 90% of the NMCC in vitro as determined by a whole-mitoplast patch-clamp recording, wherein the percent inhibition is measured 5 ms after stepping the membrane voltage from 0 to −160 mV.

In some implementations, potency of the NMCC inhibitors may be associated with higher hydrophobicity. Exemplary compounds that have been shown herein to inhibit mitochondrial conductance that can be inhibited by metformin are listed in Table 1, along with concentrations shown herein to inhibit monovalent conductance and the log octanol/water partition coefficient (log P) of the compounds.

TABLE 1
Hydrophobicity of Exemplary Inhibitors
of Mitochondrial Conductance

| Compound | Concentration (μM) | logP |
|---|---|---|
| Quinine | 150 | 3.44 |
| Proguanil | 100 | 2.53 |
| EIPA | 80 | 2.07 |
| Phenformin | 2000 | −0.83 |
| Metformin | 30000 | −1.37 |

In some embodiments, the NMCC inhibitor has a log P of about 0 or higher, about 1 or higher, about 2 or higher, about 3 or higher, about 3.5 or higher, about 3.6 or higher, 3.7 or higher, 3.8 or higher, 3.9 or higher, 4.0 or higher, 4.1 or higher, 4.2 or higher, 4.3 or higher, 4.4 or higher, 4.5 or higher, 4.6 or higher, or 4.7 or higher. In some embodiments, the log P is about 1 to 7, 6.5, 6, 5.5, 5.3, 5.1, or 5; about 2 to about 7, 6.5, 6, 5.5, 5.3, 5.1, or 5; about 3 to about 7, 6.5, 6, 5.5, 5.3, 5.1, or 5; or about 3.5 to about 7, 6.5, 6, 5.5, 5.3, 5.1, or 5; about 4 to about 7, 6.5, 6, 5.5, 5.3, 5.1, or 5; or about 4.5 to about 7, 6.5, 6, 5.5, 5.3, 5.1, or 5.

In some embodiments, the NMCC inhibitor is a compound of Formula (I):

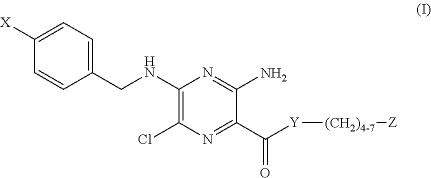

or a tautomer thereof, or a salt of any of the foregoing, wherein:

X is halo;

Y is

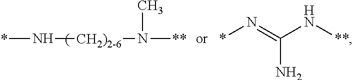

wherein:

* indicates the point of attachment to the carbonyl of the parent structure and ** indicates the point of attachment to (CH₂)₄₋₇—Y; and

Z is C₁-C₆alkyl or phenyl.

In some embodiments, the NMCC inhibitor is a compound of formula (I), wherein X is chloro or iodo.

In some embodiments, the NMCC inhibitor is a compound of formula (I), wherein Y is

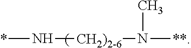

In some such embodiments, Y is of the formula

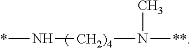

In some embodiments, Y is

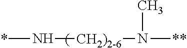

such as a Y containing four methylene groups, and X is chloro or iodo.

In some embodiments, the NMCC inhibitor is a compound of formula (I), wherein Y is

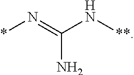

In some embodiments, the NMCC inhibitor of is a compound of formula (I), wherein Y is

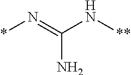

and X is chloro or iodo.

In some embodiments, the NMCC inhibitor is a compound of formula (I), wherein Z is C₁-C₆alkyl, such as methyl. In some embodiments, the NMCC inhibitor is a compound of formula (I), wherein Z is phenyl. In some embodiments, Z is C₁-C₆alkyl, such as methyl, and X is chloro or iodo. In some embodiments, Z is phenyl and X is chloro or iodo.

In some embodiments, the NMCC inhibitor is a quinoline alkaloid, a biguanide, or an amiloride. In some embodiments, the NMCC inhibitor is not a quinoline alkaloid. In some embodiments, the NMCC inhibitor of a mitochondrial conductance is not a biguanide. In some embodiments, the NMCC inhibitor of a mitochondrial conductance is not a quinoline alkaloid and not a biguanide.

In some embodiments, the NMCC inhibitor is a quinoline alkaloid. In some embodiments, the quinoline alkaloid is quinine. In some embodiments, the NMCC inhibitor is not quinine.

In some embodiments, the NMCC inhibitor is a biguanide. In some embodiments, the biguanide is metformin, proguanil, or phenformin.

In some embodiments, the NMCC inhibitor is an amiloride. In some embodiments, the amiloride is 5-(N-ethyl-N-isopropyl)amiloride (EIPA), 5-(N-methyl-N-isobutyl)amiloride (MIA), 3-amino-6-chloro-5-((4-chlorobenzyl)amino)-N-(4-(methyl(6-phenylhexyl)amino)butyl)pyrazine-2-carboxamide, (E)-3-amino-N-(amino(octylamino)methylene)-6-chloro-5-((4-iodobenzyl)amino)pyrazine-2-carboxamide, or (E)-3-amino-N-(amino((4-phenylbutyl)amino)methylene)-6-chloro-5-((4-iodobenzyl)amino)pyrazine-2-carboxamide. In some embodiments, the amiloride is not benzamil. In some embodiments, the amiloride is not EIPA, MIA, or benzamil. In some embodiments, the amiloride is 3-amino-6-chloro-5-((4-chlorobenzyl)amino)-N-(4-(methyl(6-phenylhexyl)amino)butyl)pyrazine-2-carboxamide (AA6), (E)-3-amino-N-(amino(octylamino)methylene)-6-chloro-5-((4-iodobenzyl)amino)pyrazine-2-carboxamide (5o), or (E)-3-amino-N-(amino((4-phenylbutyl)amino)methylene)-6-chloro-5-((4-iodobenzyl)amino)pyrazine-2-carboxamide (5m).

In some embodiments, the NMCC inhibitor is (E)-3-amino-N-(amino(octylamino)methylene)-6-chloro-5-((4-iodobenzyl)amino)pyrazine-2-carboxamide; (E)-3-amino-N-(amino((4-phenylbutyl)amino)methylene)-6-chloro-5-((4-iodobenzyl)amino)pyrazine-2-carboxamide; or 3-amino-6-chloro-5-((4-chlorobenzyl)amino)-N-(4-(methyl(6-phenylhexyl)amino)butyl)pyrazine-2-carboxamide; or a pharmaceutically acceptable salt thereof.

In certain embodiments, the NMCC inhibitor is a compound listed in Table 2 or Table 3. In certain embodiments, NMCC inhibitor not a compound listed in Table 2. In some embodiments, the NMCC inhibitor is a compound listed in Table 3. In some embodiments, the NMCC inhibitor is not a compound listed in Table 3. In some embodiments, the inhibitor of a mitochondrial conductance is 5o, 5m, or AA6.

Also provided are salts of compounds disclosed herein, such as pharmaceutically acceptable salts. Thus, salts, including pharmaceutically acceptable salts, of any of the compounds detailed herein, including those of Table 2 and 3 are embraced by this disclosure.

TABLE 2

| Structure | Name |
|---|---|
| 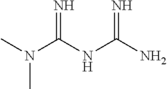 | “Metformin” |
| 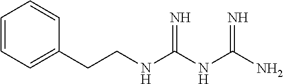 [SMILES: N=C(N)NC(=N)NCCc1ccccc1] | “Phenformin” |
| 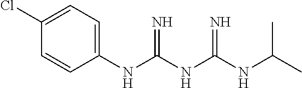 [SMILES: CC(C)NC(=N)NC(=N)Nc1ccc(Cl)cc1] | “Proguanil” |
| 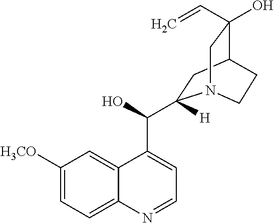 [SMILES: C=CC1(O)CN2CCC1C[C@H]2[C@H](O)c1ccnc2ccc(OC)cc12] | “Quinine” |
| 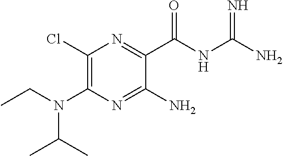 [SMILES: CCN(c1nc(N)c(C(=O)NC(=N)N)nc1Cl)C(C)C] | 5-(N-ethyl-N- isopropyl) amiloride “EIPA” |
| 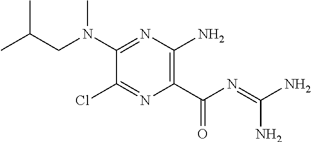 [SMILES: CC(C)CN(C)c1nc(N)c(C(=O)N=C(N)N)nc1Cl] | 5-(N-methyl- N-isobutyl) amiloride “MIA” |
| 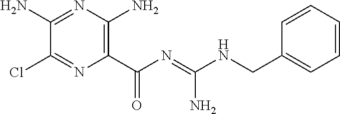 [SMILES: N/C(=N\C(=O)c1nc(Cl)c(N)nc1N)NCc1ccccc1] | benzamil |

TABLE 3

| Structure | Name |
|---|---|
| 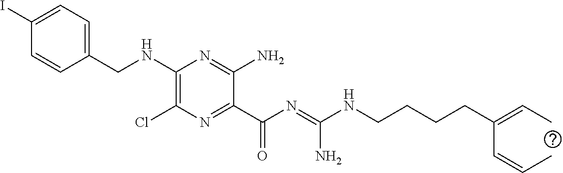 [SMILES: C/C=C\C(=C/C)CCCCN/C(N)=N/C(=O)c1nc(Cl)c(NCc2ccc(I)cc2)nc1N] | (E)-3-amino-N-(amino((4- phenylbutyl)amino)methylene)- 6-chloro-5-((4-iodobenzyl) amino)pyrazine-2-carboxamide “5m” |
| 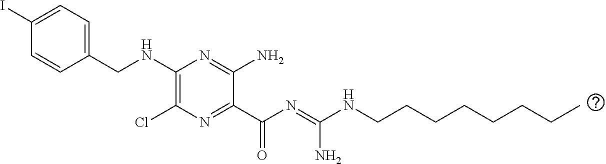 [SMILES: CCCCCCCCN/C(N)=N/C(=O)c1nc(Cl)c(NCc2ccc(I)cc2)nc1N] | (E)-3-amino-N- (amino(octylamino)methylene)- 6-chloro-5-((4-iodobenzyl) amino)pyrazine-2-carboxamide “5o” |
| 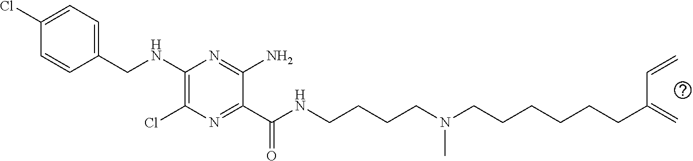 [SMILES: C=CC(=C)CCCCCCN(C)CCCCNC(=O)c1nc(Cl)c(NCc2ccc(Cl)cc2)nc1N] | 3-amino-6-chloro-5-((4- chlorobenzyl)amino)-N-(4- (methyl(6-phenylhexyl)amino) butyl)pyrazine-2-carboxamide “AA6” |

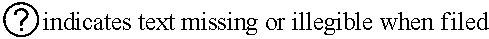

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described, such as the compounds of Table 2 or Table 3. The structures or names disclosed herein are intended to embrace all possible stereoisomers of a compound depicted. Pharmaceutical compositions comprising a compound described herein are also intended, such as a pharmaceutical composition of a specific stereochemical form, or a pharmaceutical composition comprising two or more stereochemical forms, such as in a racemic mixture.

In some embodiments, the NMCC inhibitor is a pharmaceutically acceptable salt of any of the compounds described herein. In some embodiments, a pharmaceutically acceptable salt is one or more salts of a given compound which possesses desired pharmacological activity of the free compound and which is neither biologically nor otherwise undesirable. In some embodiments, a pharmaceutically acceptable salt is one or more salts of a given compound which possesses desired pharmacological activity of the free compound and which is neither biologically nor otherwise undesirable. In some embodiments, a pharmaceutically acceptable salt includes a salt with an inorganic base, organic base, inorganic acid, organic acid, or basic or acidic amino acid.

In some implementations, the NMCC is formulated in a pharmaceutical composition. In general, the pharmaceutical composition will be a pharmaceutically acceptable formulation of the NMCC inhibitor.

In some embodiments, the pharmaceutical composition comprises an NMCC inhibitor and one or more pharmaceutically acceptable carriers. In some embodiments, the pharmaceutically acceptable carrier is a non-toxic, inert solid, semi-solid or liquid filler, diluent, encapsulating material, or formulation auxiliary of any type. Some examples of materials that can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition.

The NMCC inhibitor pharmaceutical composition may be formulated for administration via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal.

In some embodiments, the NMCC inhibitor pharmaceutical composition is suitable for administration to a human. In some embodiments, the pharmaceutical composition is suitable for administration to a mammal such as, in the veterinary context, domestic pets, and agricultural animals.

Formulations suitable for oral administration can be (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

In some embodiments, the pharmaceutical composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### IV. Methods of NMCC Inhibition and Treatment of Diseases and Conditions

Provided herein are methods of using NMCC inhibitors, such as any of the NMCC inhibitors described herein, for the modulation of mitochondrial function and the treatment of diseases associated the NMCC.

The various methods of use disclosed below will in general be directed to the administration of NMCC inhibitors to a subject. As defined above, “subject” refers to a mammal and includes, but is not limited to, human, bovine, horse, feline, canine, rodent, or primate. In one embodiment, the subject is a human. In one embodiment, the subject is a human subject at risk of or in need of treatment for a selected condition or disease. In some embodiments, the subject is a test animal, for example, an animal model of disease. In some embodiments, the subject is a veterinary subject, domestic pet, or agricultural animal. Administration to such subjects will be termed “in vivo” uses.

The various methods disclosed below will also be applicable to the treatment of mitochondria ex vivo or in vitro. As used herein, in vitro uses will refer to the administration of NMCC inhibitors to mitochondria comprising isolated mitochondria, mitochondria in tissue explants or biopsy material, and mitochondria in cultured cells, or any other ex vivo application of NMCC inhibitors.

Administration, as used herein encompasses administration to a subject by any delivery route, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal administration.

Certain implementations of the inventions disclosed herein are directed to the treatment of a class of disorders, or a specific disorder, condition or disease. Treatment, as used herein, means to ameliorate, palliate, lessen, and/or delay one or more of its symptoms, reverse or halt a pathological process, prevent, or to slow the progression of the selected condition.

In one aspect, the scope of the invention encompasses methods of inhibiting the mitochondrial inner membrane nonspecific monovalent cation conductance, or NMCC, i.e. which conductance can be inhibited by metformin. For in vivo applications, the method of inhibition encompass the administration of NMCC inhibitors to a subject. For in vitro applications, the methods of inhibition encompass the treatment or application of NMCC inhibitors to tissues, cells, or isolated mitochondria.

In one implementation, the scope of the invention encompasses a method of inhibiting the NMCC in a mitochondrion by the administration of an effective amount of an inhibitor of the NMCC. In a related implementation, the invention encompasses an inhibitor of the NMCC for use in the inhibition of the NMCC in a mitochondrion. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

In another aspect, the scope of the invention is directed to the modulation of one or more mitochondrial functions. A mitochondrial function may encompass any measure of mitochondrial activity, including, for example, respiratory activity, metabolic activity, ionic balance, pH gradient, electron transport, ATP synthesis, the production of reactive oxygen species, conductance of ions, or any other function or state of a mitochondrion.

In one implementation, the scope of the invention encompasses a method of modulating one or more mitochondrial functions in a mitochondrion by contacting the mitochondrion with an effective amount of an inhibitor of the NMCC. In a related implementation, the invention encompasses an inhibitor of the NMCC for use in the modulation of one or mitochondrial functions in a mitochondrion. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

In another aspect, the scope of the invention encompasses methods directed to the treatment of a disease or condition in a subject in need of treatment therefor, wherein the disease or condition is associated with mitochondrial function or state. In various embodiments, disease or condition comprises any disease, conditions, pathology, or metabolic process wherein one or more mitochondrial functions or states is implicated in the cause, progression, or symptoms of the condition, or wherein one or more mitochondrial functions or states is altered, dysregulated, or otherwise different from the selected function or state in healthy subjects.

In one implementation, the scope of the invention encompasses a method of treating a disease or condition associated with mitochondrial function or state in a subject in need of treatment therefor by the administration to the subject of an effective amount of an inhibitor of the NMCC. In a related implementation, the invention encompasses an inhibitor of the NMCC for use in the treatment of a disease or condition associated with mitochondrial function or state. In a related implementation, the invention encompasses the use of an inhibitor of the NMCC in the manufacture of a medicament for the treatment of a disease or condition associated with mitochondrial function or state. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

In another aspect, the scope of the invention encompasses methods directed to the treatment of diabetes. In various embodiments, diabetes, as used herein, will encompass any number of diseases or pathological processes, including, for example, diabetes mellitus, Type 1 diabetes, Type 2 diabetes, prediabetes, gestational diabetes, autoimmune destruction or impairment of pancreatic islet beta cells, reduced insulin production, insulin resistance, dysregulated glucose metabolism, progression from non-overt diabetic status to overt diabetic status, elevated fasting blood glucose concentration (e.g. greater than 130 mg/dl when fasting) and other classical symptoms such as insufficient insulin production, hyperglycemia, diabetic ketoacidosis, and other symptoms of diabetes.

In one implementation, the scope of the invention encompasses a method of treating diabetes in a subject in need of treatment therefor by the administration to the subject of an effective amount of an inhibitor of the NMCC. In a related implementation, the invention encompasses an inhibitor of the NMCC for use in the treatment of diabetes. In a related implementation, the invention encompasses the use of an inhibitor of the NMCC in the manufacture of a medicament for the treatment of diabetes. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

In another aspect, the scope of the invention is directed to the treatment of cancer by the administration of an inhibitor of the NMCC. As used herein, cancer encompasses any neoplastic condition or pathological process, for example, a solid tumor, metastatic cancer, or cancer of the blood, for example, a cancer selected from the group consisting of bladder cancer, brain cancer, breast cancer, cervical cancer, colorectal cancer, esophageal cancer, head and neck cancer, kidney cancer, lung cancer, leukemia, lymphoma, myeloma, ovarian cancer, pancreatic cancer, prostate cancer, sarcoma, and skin cancer, cancer metastasis, progression of cells from precancerous to cancer, and cancer recurrence, for example, following treatment such as radiation or surgical recession. Treatment of cancer, as used herein, may encompass achieving any therapeutic effect, including reducing tumor size, slowing cancer cell growth and proliferation, reducing the number of cancer cells, relieving one or more of the symptoms associated with the cancer, or increasing survival time.

In one implementation, the scope of the invention encompasses a method of treating cancer in a subject in need of treatment therefor by the administration to the subject of an effective amount of an inhibitor of the NMCC. In a related implementation, the invention encompasses an inhibitor of the NMCC for use in the treatment of cancer. In a related implementation, the invention encompasses the use of an inhibitor of the NMCC in the manufacture of a medicament for the treatment of cancer. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

In another aspect, the scope of the invention encompasses methods of treating a cardiac or ischemic condition. As used herein, a cardiac or ischemic condition encompasses any disease, condition, or pathological process of the heart or circulatory system, for example, any condition associated with loss of or reduced blood flow or circulation, for example those caused by vascular occlusion or constriction, insufficient blood circulation, ischemia or stroke, mini-stroke, or micro-infarct, coronary artery disease, heart attack, myocardial infarction, carotid artery disease, peripheral arterial disease, critical limb ischemia, claudication, cerebrovascular disease, reduced circulation in the brain, arterial occlusive disease in any part of the body, hypoperfusion, atherosclerosis, thrombosis, and embolism. Treatment of a cardiac or ischemic condition may encompass any number of therapeutic effects, including preventing the onset of a vascular occlusion or constriction condition; slowing or halting the progression of an ischemic condition, improving blood flow or circulation through one or more blood vessels; promoting arteriogenesis; dilating one or more blood vessels; enhancing the conductance of one or more blood vessels, improving vascular tone; or ameliorating the symptoms of a cardiac or ischemic condition.

In one implementation, the scope of the invention encompasses a method of treating a cardiac or ischemic condition in a subject in need of treatment therefor by the administration to the subject of an effective amount of an inhibitor of the NMCC. In a related implementation, the invention encompasses an inhibitor of the NMCC for use in the treatment of a cardiac or ischemic condition. In a related implementation, the invention encompasses the use of an inhibitor of the NMCC in the manufacture of a medicament for the treatment of a cardiac or ischemic condition. In various embodiments, the NMCC inhibitor may comprise any of the structures disclosed herein, for example, EIPA, a composition of Formula 1, 5m, 5o, or AA6. In various embodiments, the NMCC inhibitor may be any of: not metformin; not a quinoline alkaloid; not a biguanide; and/or not 5-(N-ethyl-N-isopropyl)amiloride (EIPA).

The dose, dosing schedule, or dosing duration of the NMCC inhibitor administered to a subject may vary with the particular the inhibitor of the mitochondrial conductance, the method of administration, and the particular disease being treated. In some embodiments, the amount of the NMCC inhibitor is a therapeutically effective amount. The effective amount of the NMCC inhibitor may in one aspect be a dose of between about 0.01 and about 100 mg/kg. Effective amounts or doses of the NMCC inhibitor may be ascertained by known methods, such as modeling, dose escalation, or clinical trials, taking into account factors, e.g., the mode or route of administration or drug delivery, the pharmacokinetics of the agent, the severity and course of the disease to be treated, the subject's health status, condition, and weight.

### V. Kits and Articles of Manufacture

The present disclosure further provides articles of manufacture comprising a matrix solution, a bath solution, and/or an NMCC inhibitor described herein or a pharmaceutical composition described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The kits may be used for any one or more of the methods or uses described herein, and, accordingly, may contain instructions for the treatment of any disease or described herein, for example for the treatment of cancer.

Kits generally comprise suitable packaging. The kits may comprise one or more containers comprising an inhibitor of a mitochondrial conductance described herein or a pharmaceutical composition described herein. Each component (if there is more than one component) can be packaged in separate containers or some components can be combined in one container where cross-reactivity and shelf life permit.

In the case of patch clamp solutions, the kits may comprise a container holding a volume of matrix solution and a volume of bath solution.

In the case of NMCC inhibitors, the kits may be in unit dosage forms, bulk packages (e.g., multi-dose packages) or sub-unit doses. For example, kits may be provided that contain sufficient dosages of an inhibitor of a mitochondrial conductance described herein or a pharmaceutical composition described herein to provide effective treatment of a subject for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to a subject.

### VI. Exemplary Embodiments

1. A method of treating a disease associated with a mitochondrial conductance in a subject, comprising administering to the subject an effective amount of an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance is not a quinoline alkaloid, a biguanide, or 5-(N-ethyl-N-isopropyl)amiloride (EIPA).

2. A method of treating a disease associated with a mitochondrial conductance in a subject, comprising administering to the subject an effective amount of an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance inhibits the mitochondrial conductance at a concentration of 10 μM or less.

3. A method of treating a disease associated with a mitochondrial conductance in a subject, comprising administering to the subject an effective amount of an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance has a log octanol/water partition coefficient (log P) of about 3.5 or higher.

4. A method of treating cancer, diabetes mellitus, or ischemia in a subject, comprising administering to the subject an effective amount of an inhibitor of a mitochondrial conductance, wherein the mitochondrial conductance is an IMM conductance for monovalent cations and/or a monovalent cation conductance that can be inhibited by metformin, and wherein the inhibitor of the mitochondrial conductance is not a quinoline alkaloid, a biguanide, or 5-(N-ethyl-N-isopropyl)amiloride (EIPA).

5. A method of treating cancer, diabetes mellitus, or ischemia in a subject, comprising administering to the subject an effective amount of an inhibitor of a mitochondrial conductance, wherein the mitochondrial conductance is an IMM conductance for monovalent cations and/or a monovalent cation conductance that can be inhibited by metformin, and wherein the inhibitor of the mitochondrial conductance inhibits the mitochondrial conductance at a concentration of 10 μM or less.

6. A method of treating cancer, diabetes mellitus, or ischemia in a subject, comprising administering to the subject an effective amount of an inhibitor of a mitochondrial conductance, wherein the mitochondrial conductance is an IMM conductance for monovalent cations and/or a monovalent cation conductance that can be inhibited by metformin, and wherein the inhibitor of the mitochondrial conductance has a log octanol/water partition coefficient (log P) of about 3.5 or higher.

7. The method of any one of embodiments 4-6, wherein the cancer is a solid cancer.

8. The method of any one of embodiments 4-6, wherein the cancer is a cancer of the blood.

9. The method of any one of embodiments 4-6, wherein the diabetes mellitus is type II diabetes mellitus.

10. The method of any one of embodiments 4-6, wherein the ischemia is a cardiac ischemia.

11. The method of any one of embodiments 1-10, wherein the subject is a human.

12. The method of any one of embodiments 1-10, wherein the subject is a canine.

13. A method of inhibiting a mitochondrial conductance, comprising contacting a mitochondrial membrane with an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance is not a quinoline alkaloid, a biguanide, or 5-(N-ethyl-N-isopropyl)amiloride (EIPA).

14. A method of inhibiting a mitochondrial conductance, comprising contacting a mitochondrial membrane with an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance inhibits the mitochondrial conductance at a concentration of 10 μM or less.

15. A method of inhibiting a mitochondrial conductance, comprising contacting a mitochondrial membrane with an inhibitor of the mitochondrial conductance, wherein the mitochondrial conductance is an IMM conductance for monovalent cations and/or a monovalent cation conductance that can be inhibited by metformin, and wherein the inhibitor of the mitochondrial conductance has a log octanol/water partition coefficient (log P) of about 3.5 or higher.

16. The method of any of embodiments 1-15, wherein the mitochondrial conductance can be inhibited by 30 mM metformin.

17. The method of any of embodiments 1-16, wherein the mitochondrial conductance can be inhibited in vitro by 30 mM metformin.

18. The method of any one of embodiments 1, 3-4, 6-13, or 15-17, wherein the inhibitor of the mitochondrial conductance inhibits the mitochondrial conductance at a concentration of 10 μM or less.

19. The method of any one of embodiments 1-2, 4-5, 7-14, or 16-17, wherein the inhibitor of the mitochondrial conductance has a log octanol/water partition coefficient (log P) of about 3.5 or higher.

20. The method of any one of embodiments 1-19, wherein the inhibitor of the mitochondrial conductance is an amiloride.

21. The method of any one of embodiments 1-20, wherein the inhibitor of the mitochondrial conductance is a compound of Formula (I):

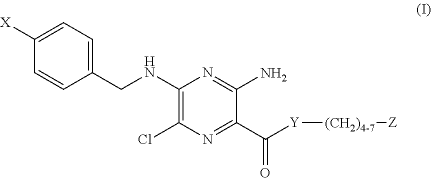

or a tautomer thereof, or a salt of any of the foregoing, wherein:

X is halo;

Y is

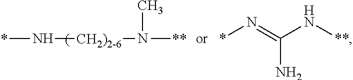

wherein:
* indicates the point of attachment to the carbonyl of the parent structure and ** indicates the point of attachment to (CH₂)₄₋₇—Y; and

Z is C₁-C₆alkyl or phenyl.

22. The method of embodiment 20, wherein the amiloride is not 5-(N-methyl-N-isobutyl)amiloride (MIA) or benzamil.

23. The method of any one of embodiments 1-22, wherein the inhibitor of the mitochondrial conductance is

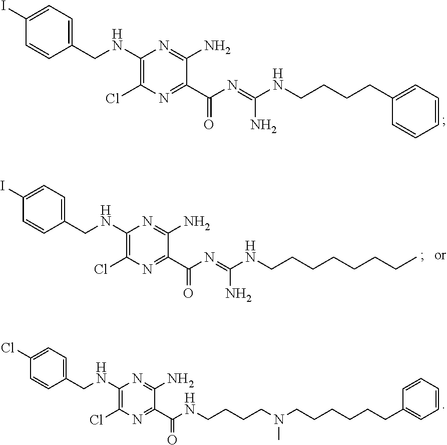 [SMILES: N/C(=N\C(=O)c1nc(Cl)c(NCc2ccc(I)cc2)nc1N)NCCCCc1ccccc1 ; CCCCCCCCN/C(N)=N/C(=O)c1nc(Cl)c(NCc2ccc(I)cc2)nc1N ; CN(CCCCCCc1ccccc1)CCCCNC(=O)c1nc(Cl)c(NCc2ccc(Cl)cc2)nc1N]

24. A method of screening a test compound for inhibition of a mitochondrial conductance, wherein the mitochondrial conductance is an IMM conductance for monovalent cations and/or a monovalent cation conductance that can be inhibited by metformin, comprising: contacting a mitochondrial membrane with the test compound; and measuring a current change or a voltage change across the mitochondrial membrane in the presence of a monovalent cation.

25. The method of embodiment 24, wherein the mitochondrial conductance can be inhibited by 30 mM metformin.

26. The method of embodiment 24 or 25, wherein the mitochondrial conductance can be inhibited in vitro by 30 mM metformin.

27. The method of any of embodiments 24-26, further comprising applying a voltage to the mitochondrial membrane.

28. The method of embodiment 27, wherein the voltage applied is about −200 mV to about +200 mV.

29. The method of any one of embodiments 24-28, wherein the monovalent cation is a potassium ion or a sodium ion.

30. The method of any one of embodiments 24-29, further comprising exposing the mitochondrial membrane to heat.

31 The method of any one of embodiments 24-30, wherein the mitochondrial membrane is an inner mitochondrial membrane (IMM).

32. The method of embodiment 31, wherein the IMM is part of a mitoplast.

33. The method of any one of embodiments 24-32, further comprising rupturing an outer mitochondrial membrane (OMNI).

34. The method of embodiments 33, wherein the rupturing comprises applying a French press to a mitochondrion.

35. The method of any one of embodiments 24-34, further comprising contacting the mitochondrial membrane with a recording electrode and rupturing a portion of the mitochondrial membrane contacted by the recording electrode.

36. The method of embodiment 35, wherein rupturing the portion of the mitochondrial membrane contacted by the recording electrode comprises applying one or more electrical pulses to the portion of the mitochondrial membrane contacted by the recording electrode.

37. The method of any one of embodiments 24-36, wherein the test compound is applied to an outer surface of the mitochondrial membrane.

38. The method of any one of embodiments 24-37, wherein the monovalent cation is applied to an inner surface of the mitochondrial membrane, an outer surface of the mitochondrial membrane, or both an inner surface of the mitochondrial membrane and an outer surface of the mitochondrial membrane.

39. The method of any one of embodiment 24-38, further comprising identifying the test compound as an inhibitor of the mitochondrial conductance.

40. An inhibitor identified by the method of embodiment 39.

Additional exemplary embodiments include:
E1. A method of measuring the inner mitochondrial membrane nonselective monovalent cation conductance, comprising the steps of: applying voltage to induce the flow of monovalent cations across the inner mitochondrial membrane; and obtaining measurements that indicate the magnitude of monovalent cation current across the inner mitochondrial membrane; and wherein the MCU activity in the inner mitochondrial membrane is inhibited.
E2. The measurement method of exemplary embodiment E1, further comprising obtaining a mitochondrion from a selected source.
E3. The measurement method of exemplary embodiment E1 or E2, further comprising treating mitochondrion to make the inner mitochondrial membrane thereof accessible, wherein the treated mitochondrion is in a bath solution.
E4. The measurement method of any one of exemplary embodiments E1-E3, further comprising contacting a patch clamp recording electrode to the inner mitochondrial membrane and forming a seal therewith, wherein the patch clamp electrode comprises a matrix solution; and wherein a reference electrode is present in the bath solution.
E5. The measurement method of any one of exemplary embodiments E1-E4, wherein the measurements are obtained by readout of the recording electrode.
E6. The measurement method of any one of exemplary embodiments E1-E5, wherein the inner mitochondrial membrane nonselective monovalent cation conductance is a conductance that can be inhibited by metformin.
E7. The measurement method of any of the foregoing exemplary embodiments, wherein the inner mitochondrial membrane nonselective monovalent cation conductance is a conductance that be inhibited by 30 mM metformin.
E8. The measurement method of any of the foregoing exemplary embodiments, wherein the treatment to make the inner mitochondrial membrane accessible is the formation of a mitoplast.
E9. The measurement method of any of the foregoing exemplary embodiments, wherein the bath solution comprises: a monovalent cation; a chelator which removes substantially all divalent cations from the solution; a buffer; a sugar or sugar alcohol; wherein the pH of the bath solution is between about 6.5 and 9; and wherein the osmolality of the bath solution is between about 250 to about 500 mmol/kg.
E10. The measurement method of any of the foregoing exemplary embodiments, wherein the bath solution comprises a monovalent cation comprising Na, K, Li, Cs, TMA, or a mixture of the foregoing.
E11. The measurement method of any of the foregoing exemplary embodiments, wherein the monovalent cation is provided to the bath solution in the form of a gluconate salt.
E12. The measurement method of any of the foregoing exemplary embodiments, wherein the bath solution does not comprise chloride.
E13. The measurement method of any of the foregoing exemplary embodiments, wherein the bath solution comprises a chelator which removes substantially all divalent cations from the solution is selected from the group consisting of: (1,2-bis(o-aminophenoxy)ethane-N,N,N′,N′-tetraacetic acid (BAPTA), deferoxamine, diethylenetriaminepentaacetic acid (DTPA), 2,3-dimercapto-1-propanesulfonic acid (DMPS), dimercaptosuccinic acid (DMSA), polyaspartic acid ethylenediamine-N,N′-disuccinic acid (EDDS), ethylenediaminetetraacetic acid (EDTA), ethylene glycol-bis(β-aminoethyl ether)-N,N,N′,N′-tetraacetic acid) (EGTA), methylglycinediacetic acid, L-glutamic acid N,N-diacetic acid, tetrasodium salt (GLDA), iminodisuccinic acid (IDS), or lipoic acid (LA), or a mixture of any of the foregoing.
E14. The measurement method of any of the foregoing exemplary embodiments, wherein the chelator of the bath solution is a mixture of EDTA and EGTA.
E15. The measurement method of any of the foregoing exemplary embodiments, wherein the bath solution comprises a buffer, wherein the buffer is selected from the group consisting of ACES, ADA, AMPB, AMPSO, BES, CABS, CHES, DIPSO, HEPBS, HEPES, HEPPS, HEPPSO, MOBS, MOPS, MOPSO, PIPES, POPSO, TAPS, TAPSO, TES, tricine, triethanolamine, and tris.
E16. The measurement method of any of the foregoing exemplary embodiments, wherein the buffer of the bath solution is HEPES.
E17. The measurement method of any of the foregoing exemplary embodiments, wherein the pH of the bath solution is between about 7.0 to 7.5.
E18. The measurement method of any of the foregoing exemplary embodiments, wherein the osmolality of the bath solution is between about 330-450 mmol/kg.
E19. The measurement method of any of the foregoing exemplary embodiments, wherein the bath solution comprises a sugar or sugar alcohol.
E20. The measurement method of any of the foregoing exemplary embodiments, wherein the sugar or sugar alcohol comprises sucrose.
E21. The measurement method of any of the foregoing exemplary embodiments, wherein the matrix solution comprises a monovalent cation; a chelator which removes substantially all divalent cations from the solution; a buffer; a sugar or sugar alcohol; wherein the pH of the bath solution is between about 6.5 and 9; and wherein the osmolality of the bath solution is between about 250 to about 500 mmol/kg.
E22. The measurement method of any of the foregoing exemplary embodiments, wherein the matrix solution comprises a monovalent cation comprising Na, K, Li, Cs, TMA, or a mixture of the foregoing.
E23. The measurement method of any of the foregoing exemplary embodiments, wherein the monovalent cation is provided to the matrix solution in the form of a gluconate salt.
E24. The measurement method of any of the foregoing exemplary embodiments, wherein the matrix solution does not comprise chloride.
E25. The measurement method of any of the foregoing exemplary embodiments, wherein the matrix solution comprises a chelator which removes substantially all divalent cations from the solution.
E26. The measurement method of any of the foregoing exemplary embodiments, wherein the matrix solution chelator is selected from the group consisting of: (1,2-bis(o-aminophenoxy)ethane-N,N,N′,N′-tetraacetic acid (BAPTA), deferoxamine, diethylenetriaminepentaacetic acid (DTPA), 2,3-dimercapto-1-propanesulfonic acid (DMPS), dimercaptosuccinic acid (DMSA), polyaspartic acid ethylenediamine-N,N′-disuccinic acid (EDDS), ethylenediaminetetraacetic acid (EDTA), ethylene glycol-bis(β-aminoethyl ether)-N,N,N′,N′-tetraacetic acid) (EGTA), methylglycinediacetic acid, L-glutamic acid N,N-diacetic acid, tetrasodium salt (GLDA), iminodisuccinic acid (IDS), or lipoic acid (LA), or a mixture of any of the foregoing.
E27. The measurement method of any of the foregoing exemplary embodiments, wherein the matrix solution chelator is a mixture of EDTA and EGTA.
E28. The measurement method of any of the foregoing exemplary embodiments, wherein the matrix solution comprises a buffer, wherein the buffer is selected from the group consisting of ACES, ADA, AMPB, AMPSO, BES, CABS, CHES, DIPSO, HEPBS, HEPES, HEPPS, HEPPSO, MOBS, MOPS, MOPSO, PIPES, POPSO, TAPS, TAPSO, TES, tricine, triethanolamine, and tris.
E29. The measurement method of any of the foregoing exemplary embodiments, wherein the buffer of the matrix solution is HEPES.
E30. The measurement method of any of the foregoing exemplary embodiments, wherein the pH of the matrix solution is between about 7.0 to 7.5.
E31. The measurement method of any of the foregoing exemplary embodiments, wherein the osmolality of the matrix solution is between about 330-450 mmol/kg.
E32. The measurement method of any of the foregoing exemplary embodiments, wherein the matrix solution comprises a sugar or sugar alcohol.
E33. The measurement method of any of the foregoing exemplary embodiments, wherein the sugar or sugar alcohol comprises sucrose.
E34. The measurement method of any of the foregoing exemplary embodiments, wherein the MCU conductance of the IMM is inhibited by the use of measurement conditions wherein calcium is absent.
E35. The measurement method of any of the foregoing exemplary embodiments, wherein the matrix and bath solution do not comprise calcium.
E36. The measurement method of any of the foregoing exemplary embodiments, wherein the MCU conductance of the IMM is inhibited by the application of an MCU inhibitor.
E37. The measurement method of any of the foregoing exemplary embodiments, wherein the MCU conductance of the IMM is inhibited by the use of mitochondria comprising mutations that reduce the activity of the MCU.
E38. The measurement method of any of the foregoing exemplary embodiments, wherein the mitochondrion is heated prior to and/or during the measurement process.
E39. The measurement method of any of the foregoing exemplary embodiments, wherein the mitochondrion is heated by use of a bath solution having a temperature between 30 and 37° C.
E40. The measurement method of any of the foregoing exemplary embodiments, wherein the mitochondrion is cooled for a period of time prior to performing the measurement.
E41. The measurement method of any of the foregoing exemplary embodiments, wherein the bath solution is placed on ice for a period of 15-30 minutes prior to the measurement.
E42. The measurement method of any of the foregoing exemplary embodiments, wherein the bath solution and/or matrix solution comprises an inhibitor or putative inhibitor of the inner mitochondrial membrane nonselective monovalent cation conductance.
E43. A method of identifying inhibitor of the inner mitochondrial membrane nonselective monovalent cation conductance, comprising: performing a first measurement of monovalent cation flux across the inner mitochondrial membrane by any of the foregoing measurement methods; wherein the method comprises contacting the inner mitochondrial membrane with a putative inhibitor of the inner mitochondrial membrane nonselective monovalent cation conductance.
E44. The method of exemplary embodiment E43, further comprising performing a second measurement of monovalent cation flux across the inner mitochondrial membrane by the method of any the foregoing measurement methods; wherein second measurement does not comprise contacting the inner mitochondrial membrane with a putative inhibitor of the inner mitochondrial membrane nonselective monovalent cation conductance.
E45. The method of exemplary embodiment E44, wherein the second measurement is performed prior to the first measurement.
E46. The method of exemplary embodiment E44 or E45, further comprising comparing the first and second measurements.
E47. The method of exemplary embodiment E46, wherein, if a reduction in the measured inner mitochondrial membrane nonselective monovalent cation conductance is observed in the first measurement, the selected composition of matter is identified as an inhibitor of the inner mitochondrial membrane nonselective monovalent cation conductance.
E48. An inhibitor of the mitochondrial inner membrane nonspecific monovalent cation conductance for use a method of inhibiting the mitochondrial inner membrane nonspecific monovalent cation conductance in a mitochondrion.
E49. The inhibitor of the mitochondrial inner membrane nonspecific monovalent cation conductance of exemplary embodiment E48, wherein the mitochondrion is in vivo.
E50. The inhibitor of the mitochondrial inner membrane nonspecific monovalent cation conductance of exemplary embodiment E48, wherein the mitochondrion is in vitro.
E51. An inhibitor of the mitochondrial inner membrane nonspecific monovalent cation conductance for use in a method of modulating one or mitochondrial functions in the mitochondria of a subject.
E52. An inhibitor of the mitochondrial inner membrane nonspecific monovalent cation conductance for use in a method of treating a disease or condition wherein the disease or condition is associated with mitochondrial function or state.
E53. An inhibitor of the mitochondrial inner membrane nonspecific monovalent cation conductance for use in a method of treating a disease or condition wherein the disease or condition is associated with mitochondrial function or state wherein the inhibitor is not metformin.
E54. An inhibitor of the mitochondrial inner membrane nonspecific monovalent cation conductance for use in a method of treating diabetes or a diabetes related condition.
E55. The inhibitor of exemplary embodiments E54, wherein the diabetes or diabetes related condition is one or more of diabetes mellitus, Type 1 diabetes, Type 2 diabetes, prediabetes, gestational diabetes, autoimmune destruction or impairment of pancreatic islet beta cells, reduced insulin production, insulin resistance, dysregulated glucose metabolism, progression from non-overt diabetic status to overt diabetic status, elevated fasting blood glucose concentration hyperglycemia, and diabetic ketoacidosis.
E56. The inhibitor of exemplary embodiments E54 or E55, wherein the inhibitor of the mitochondrial inner membrane nonspecific monovalent cation conductance the inhibitor is not metformin.
E57. An inhibitor of the mitochondrial inner membrane nonspecific monovalent cation conductance for use in a method of treating cancer.
E58. The inhibitor of the mitochondrial inner membrane nonspecific monovalent cation conductance for use in a method of treating cancer, wherein the cancer comprises any of a solid tumor, metastatic cancer, cancer of the blood, bladder cancer, brain cancer, breast cancer, cervical cancer, colorectal cancer, esophageal cancer, head and neck cancer, kidney cancer, lung cancer, leukemia, lymphoma, myeloma, ovarian cancer, pancreatic cancer, prostate cancer, sarcoma, and skin cancer, cancer metastasis, progression of cells from precancerous to cancer, and cancer recurrence.
E59. The inhibitor of exemplary embodiments E57 or E58, wherein the inhibitor is not metformin.
E60. An inhibitor of the mitochondrial inner membrane nonspecific monovalent cation conductance for use in a method of treating a cardiac or ischemic condition.
E61. The inhibitor of exemplary embodiment E60, wherein the cardiac or ischemic condition encompasses any of vascular occlusion or constriction, insufficient blood circulation, ischemia or stroke, mini-stroke, or micro-infarct, coronary artery disease, heart attack, myocardial infarction, carotid artery disease, peripheral arterial disease, critical limb ischemia, claudication, cerebrovascular disease, reduced circulation in the brain, arterial occlusive disease, hypoperfusion, atherosclerosis, thrombosis, and embolism.
E62. The inhibitor of embodiment E60 or E61, wherein the inhibitor is not metformin.
E63. A method of inhibiting the mitochondrial inner membrane nonspecific monovalent cation conductance in a mitochondrion, wherein such conductance can be inhibited by metformin, comprising administering to a mitochondrion of an effective amount of an inhibitor of the mitochondrial inner membrane nonspecific monovalent cation conductance.
E64. The method of exemplary embodiment E63, wherein the mitochondrion is in vivo.
E65. The method of exemplary embodiment E63 or E64, wherein the mitochondrion is in vitro.
E66. A method of modulating one or mitochondrial functions in the mitochondria of a subject, comprising administering to the subject an effective amount of an inhibitor of the mitochondrial inner membrane nonspecific monovalent cation conductance.
E67. A method of treating a disease or condition wherein the disease or condition is associated with mitochondrial function or state, comprising administering to the subject an effective amount of an inhibitor of the mitochondrial inner membrane nonspecific monovalent cation conductance.
E68. A method of treating diabetes or a diabetes related condition in a subject in need of treatment therefor, comprising administering to the subject an effective amount of an inhibitor of the mitochondrial inner membrane nonspecific monovalent cation conductance.
E69. The method of exemplary embodiment E68, wherein the diabetes or diabetes related condition is one or more of diabetes mellitus, Type 1 diabetes, Type 2 diabetes, prediabetes, gestational diabetes, autoimmune destruction or impairment of pancreatic islet beta cells, reduced insulin production, insulin resistance, dysregulated glucose metabolism, progression from non-overt diabetic status to overt diabetic status, elevated fasting blood glucose concentration hyperglycemia, and diabetic ketoacidosis.
E70. The method of exemplary embodiment E68 or E69, wherein the inhibitor of the mitochondrial inner membrane nonspecific monovalent cation conductance is not metformin.
E71. A method of treating cancer in a subject in need of treatment therefor, comprising administering to the subject an effective amount of an inhibitor of the mitochondrial inner membrane nonspecific monovalent cation conductance.
E72. The method of exemplary embodiment E71, wherein the cancer comprises any of a solid tumor, metastatic cancer, cancer of the blood, bladder cancer, brain cancer, breast cancer, cervical cancer, colorectal cancer, esophageal cancer, head and neck cancer, kidney cancer, lung cancer, leukemia, lymphoma, myeloma, ovarian cancer, pancreatic cancer, prostate cancer, sarcoma, and skin cancer, cancer metastasis, progression of cells from precancerous to cancer, and cancer recurrence.
E73. The method of exemplary embodiment E71 or E72, wherein the inhibitor of the mitochondrial inner membrane nonspecific monovalent cation conductance is not metformin.
E74. A method of treating a cardiac or ischemic condition in a subject in need of treatment therefor, comprising administering to the subject an effective amount of an inhibitor of the mitochondrial inner membrane nonspecific monovalent cation conductance.
E75. The method of exemplary embodiment E74, wherein the cardiac or ischemic condition encompasses any of vascular occlusion or constriction, insufficient blood circulation, ischemia or stroke, mini-stroke, or micro-infarct, coronary artery disease, heart attack, myocardial infarction, carotid artery disease, peripheral arterial disease, critical limb ischemia, claudication, cerebrovascular disease, reduced circulation in the brain, arterial occlusive disease, hypoperfusion, atherosclerosis, thrombosis, and embolism.
E76. The method of exemplary embodiment E74 or E75, wherein the inhibitor of the mitochondrial inner membrane nonspecific monovalent cation conductance is not metformin.
E77. The method or inhibitor of any of the foregoing, wherein the mitochondrial inner membrane nonspecific monovalent cation conductance is a conductance that can be inhibited by metformin.
E78. The method or inhibitor of any of the foregoing, wherein the mitochondrial inner membrane nonspecific monovalent cation conductance is a conductance that can be inhibited by metformin at a concentration of 30 mM.
E79. The method or inhibitor of any of the foregoing, wherein the inhibitor of the mitochondrial inner membrane nonspecific monovalent cation conductance inhibits the mitochondrial inner membrane nonspecific monovalent cation conductance at a concentration of 10 μM or less.
E80. The method or inhibitor of any of the foregoing, wherein the inhibitor of the mitochondrial inner membrane nonspecific monovalent cation conductance has a log octanol/water partition coefficient (log P) of about 3.5 or higher.
E81. The method or inhibitor of any of the foregoing, wherein the subject is a human.
E82. The method or inhibitor of any of the foregoing, wherein the inhibitor of the mitochondrial inner membrane nonspecific monovalent cation conductance is an amiloride.
E83. The method or inhibitor of any of the foregoing, wherein the inhibitor of the mitochondrial inner membrane nonspecific monovalent cation conductance is 5-(N-methyl-N-isobutyl)amiloride.
E84. The method or inhibitor of any of the foregoing, wherein the inhibitor of the mitochondrial inner membrane nonspecific monovalent cation conductance is benzamil.
E85. The method or inhibitor of any of the foregoing, wherein the amiloride is not 5-(N-methyl-N-isobutyl)amiloride or benzamil.
E86. The method or inhibitor of any of the foregoing, wherein the inhibitor of the mitochondrial inner membrane nonspecific monovalent cation conductance is a compound of Formula (I):

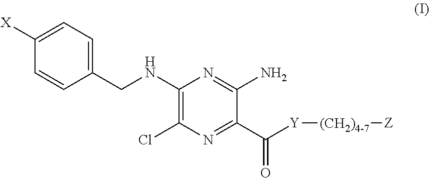

or a tautomer thereof, or a salt of any of the foregoing, wherein:

X is halo;

Y is

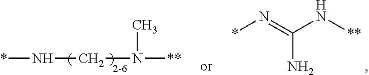

wherein:

* indicates the point of attachment to the carbonyl of the parent structure and ** indicates the point of attachment to (CH₂)₄₋₇—Y; and

Z is C₁-C₆alkyl or phenyl.
E87. The method or inhibitor of any of the foregoing, wherein the inhibitor of the mitochondrial inner membrane nonspecific monovalent cation conductance is

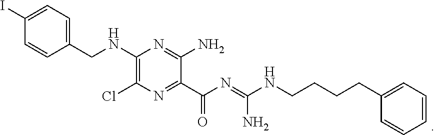 [SMILES: N/C(=N\C(=O)c1nc(Cl)c(NCc2ccc(I)cc2)nc1N)NCCCCc1ccccc1]

E88. The method or inhibitor of any of the foregoing, wherein the inhibitor of the mitochondrial inner membrane nonspecific monovalent cation conductance is

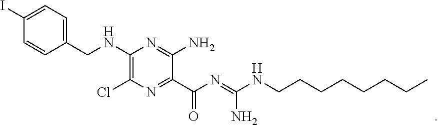 [SMILES: CCCCCCCCN/C(N)=N/C(=O)c1nc(Cl)c(NCc2ccc(I)cc2)nc1N]

E89. The method or inhibitor of any of the foregoing, wherein the inhibitor of the mitochondrial inner membrane nonspecific monovalent cation conductance is

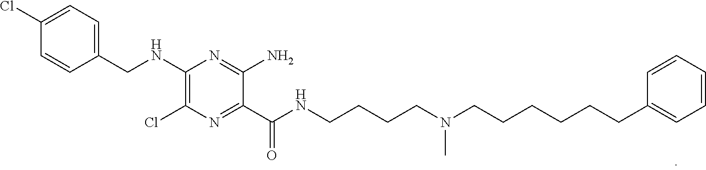 [SMILES: CN(CCCCCCc1ccccc1)CCCCNC(=O)c1nc(Cl)c(NCc2ccc(Cl)cc2)nc1N]

E90. The method or inhibitor of any of the foregoing, wherein the inhibitor of the mitochondrial inner membrane nonspecific monovalent cation conductance is metformin.
E91. The method or inhibitor of any of the foregoing, wherein the inhibitor of the mitochondrial inner membrane nonspecific monovalent cation conductance is any of: a quinoline alkaloid; a biguanide; and 5-(N-ethyl-N-isopropyl)amiloride.
E92. The method or inhibitor of any of the foregoing, wherein the inhibitor of the mitochondrial inner membrane nonspecific monovalent cation conductance is any of: not a quinoline alkaloid; not a biguanide; and not 5-(N-ethyl-N-isopropyl)amiloride.

### EXAMPLES

The following examples are included for illustrative purposes only and are not intended to limit the scope of the invention.

### Example 1: Mitoplast Patch-Clamp

A. Isolation of Mitoplasts for Patch-Clamp Recording

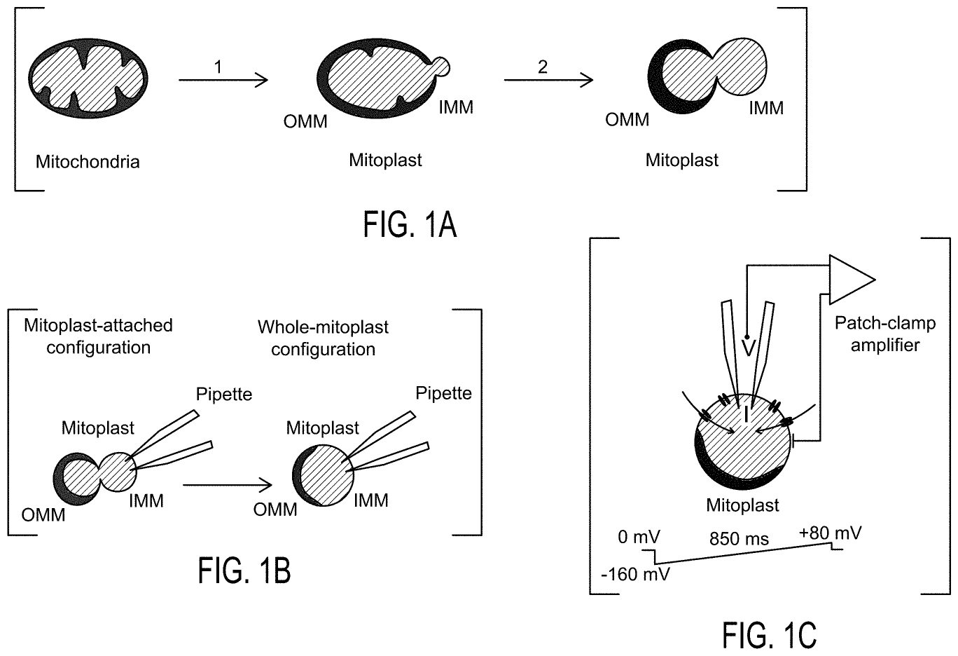

Tissue was isolated, rinsed and homogenized in ice-cold medium containing 250 mM sucrose, 10 mM HEPES, and 1 mM EGTA (pH adjusted to 7.25 with KOH), using a glass grinder with six slow strokes of a Teflon pestle rotating at 280 (soft tissues) or 600 (fibrous tissues) rotations per minute. The homogenate was centrifuged at 700 g for 5-10 min to pellet nuclei and unbroken cells. For some tissues, the first nuclear pellet was resuspended in the same solution and homogenized again to increase the yield of mitochondria. Mitochondria were collected by centrifugation of the supernatant at 8,500 g for 10 min. Mitochondria were suspended in a solution containing 140 mM sucrose, 440 mM D-mannitol, 5 mM HEPES, and 1 mM EGTA (pH adjusted to 7.2 with KOH), and then subjected to a French press at 2,000 psi to rupture the outer mitochondrial membrane (OMNI) and release the inner mitochondrial membrane (IMM) to form mitoplasts. Mitoplasts were pelleted at 10,500 g for 15 min and resuspended for storage in 500 ml of solution containing 750 mM KCl, 100 mM HEPES and 1 mM EGTA (pH adjusted to 7.2 with KOH) to further release the IMM from the OMNI, see FIG. 1A. The further release of the IMM from the OMNI caused the mitoplasts assume an 8-shaped form. Mitochondria and mitoplasts were prepared at 0-4° C. and stored on ice for up to 5 h. Tissues were isolated from a mitochondrial calcium uniporter (MCU) knock-out mouse unless otherwise indicated.

B. Patch Clamp Recordings

A glass pipette recording electrode was used to form a gigaohm seal with the IMM in a bath solution containing 150 mM KCl, 10 mM HEPES, and 1 mM EGTA, pH 7.2 (adjusted with KOH). Capacitive transients were completely compensated right after the seal was formed. The patch of the IMM attached to the glass pipette recording electrode was then ruptured by high-amplitude voltage pulses (200-500 mV). When the IMM patch was ruptured, the IMM was nearly completely released from the OMM, and the mitoplast acquired a round shape, FIG. 1B.

A patch-clamp amplifier was used to induce a current as shown in FIG. 1C. Currents were recorded using an Axopatch 200B amplifier (Molecular Devices). The current was dropped to −160 mV and then ramped up for 850 mS to +80 mV. All voltages indicated correspond to the mitochondrial matrix in respect to the bath (cytosol). Mitoplast conductance was normalized to the membrane capacitance (Cm) in all tissues examined. Current amplitudes for histograms were measured within 5 mS after stepping the membrane from 0 to −160 mV and were normalized to the total current. Whole-mitoplast control currents were recorded in a bath solution containing 150 mM HEPES, 1.5 mM EGTA, pH 7.0 adjusted with Tris base, osmolarity to 300 mmol/kg with sucrose.

### Example 2: Mitoplast Patch-Clamp with Monovalent Cations

When monovalent cations were used in the bath surrounding the mitoplast or in the pipette with the mitochondrial patch-clamp described in Example 1, monovalent cation conductance could be seen.

A. Voltage-Step Protocol

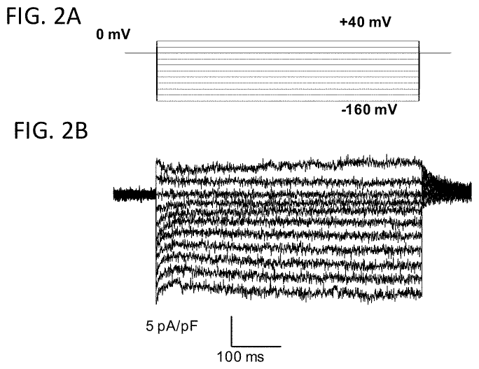

A mitoplast patch-clamp was performed as described in Example 1, except that the voltage-step protocol was applied as shown in FIG. 2A. The pipette solution contained 110 mM Na-gluconate, 5 mM EGTA, 40 mM HEPES, 5 mM EDTA, and 1 mM NaCl and was tonicity adjusted to ˜370 mmol/kg with sucrose and pH adjusted to pH 7 with 1 M NaOH. The bath solution contained 110 mM Na-gluconate, 5 mM EGTA, 40 mM HEPES, and 5 mM EDTA and was pH adjusted to pH 7 with 1 M NaOH. The resulting conductance measured is shown in FIG. 2B and was proportional to the voltage applied.

B. Conductance of Various Monovalent Cations

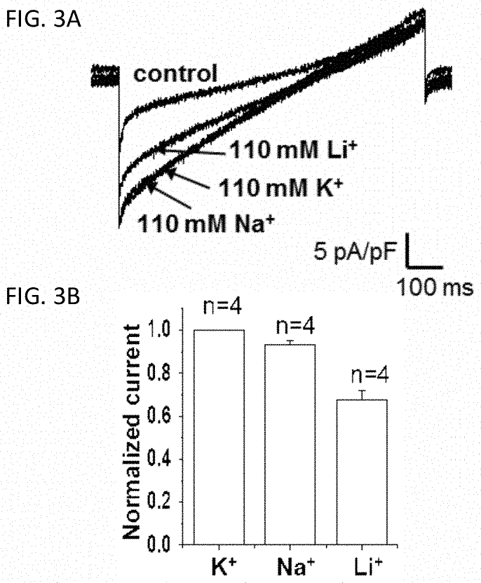

Mitoplast patch-clamp recordings were performed as described in Example 1 to compare the conductance of the monovalent cations Na⁺, K⁺, and Li. For the Na⁺ condition, the pipette solution contained 110 mM Na-gluconate, 5 mM EGTA, 40 mM HEPES, 5 mM EDTA, and 1 mM NaCl and was tonicity adjusted to ˜350-370 mmol/kg with sucrose and pH adjusted to pH 7 with 1 M NaOH, and the bath solution contained 110 mM Na-gluconate, 5 mM EGTA, 40 mM HEPES, and 5 mM EDTA and tonicity adjusted to ˜300 mmol/kg with sucrose and pH adjusted to pH 7 with 1 M NaOH. For the K⁺ condition, the pipette solution contained 110 mM K-gluconate, 5 mM EGTA, 40 mM HEPES, 5 mM EDTA, and 1 mM KCl and was tonicity adjusted to ˜350-370 mmol/kg with sucrose and pH adjusted to pH 7 with 1 M KOH and the bath solution contained 110 mM K-gluconate, 5 mM EGTA, 40 mM HEPES, and 5 mM EDTA and tonicity adjusted to ˜300 mmol/kg with sucrose and pH adjusted to pH 7 with 1 M KOH. For the Li⁺ condition, the pipette solution contained 110 mM Li-gluconate, 1 mM EGTA, 40 mM HEPES, 5 mM EDTA, and 1 mM NaCl and was tonicity adjusted to ˜350-370 mmol/kg with sucrose and pH adjusted to pH 7 with D Gluconic Acid, and the bath solution contained 110 mM Li-gluconate, 1 mM EGTA, 40 mM HEPES, and 5 mM EDTA and was pH adjusted to pH 7 with D Gluconic Acid. Exemplary resulting conductances measured for the three conditions are shown in FIG. 3A. The current amplitudes measured at 5 mS after stepping the membrane from 0 to −160 mV and normalized to the total current are shown in FIG. 3B. Conductance was able to be seen for all three ions.

C. The Effect of pH on Monovalent Cation Conductance

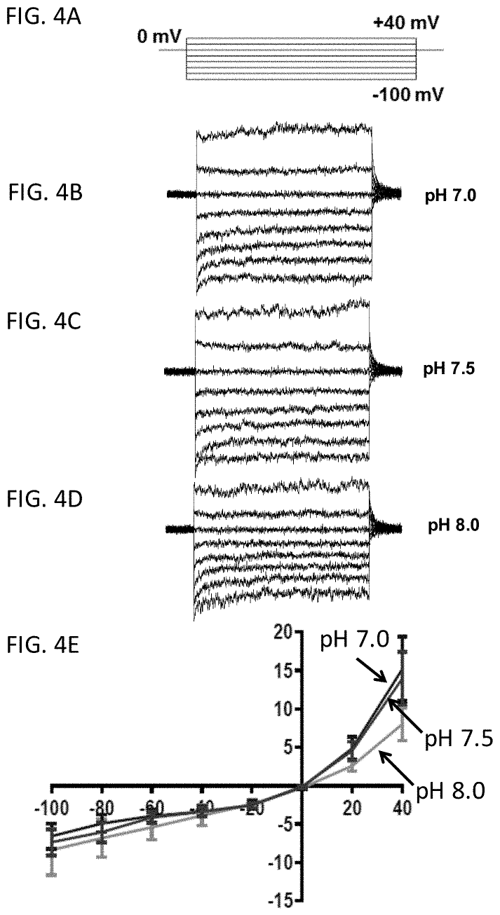

Mitoplast patch-clamp recordings were performed as described in Example 1 to determine the effect of pH on the conductance of the exemplary monovalent cation Na⁺, except that the voltage-step protocol was applied as shown in FIG. 4A. For all conditions, the pipette solution contained 125 mM Na-gluconate, 1 mM EGTA, 40 mM HEPES, 1 mM EDTA, and 1 mM NaCl and was pH adjusted to 7.5 and tonicity adjusted to ˜350-370 mmol/kg with sucrose, and the bath solution contained 125 mM Na-gluconate, 1 mM EGTA, 40 mM HEPES, and 1 mM EDTA and was tonicity adjusted to ˜300 mmol/kg with sucrose. The solutions were pH adjusted to pH 7.0, 7.5, or 8.0 with 1 M NaOH. The resulting conductances are shown in FIGS. 4B-4D and a graphical depiction of resulting conductances (I/V plot) seen with the different pH assays is shown in FIG. 4E. The pH did not affect the membrane reversal potential, indicating no permeation or transport of H⁺ or OH⁻ through the conductance.

D. Monovalent Cation Conductance in Mitoplasts from Different Tissues

To determine the presence of the monovalent cation conductance in mitoplasts in different tissue types, mice mitoplasts were prepared from heart, skeletal muscle, liver, kidney, or brown fat of adult (3-6-week-old) wild-type C56BL/6 mice or C56BL/6 mice deficient for uncoupling protein 1 (UCP1−/−) for brown fat mitoplasts as described in Example 1.

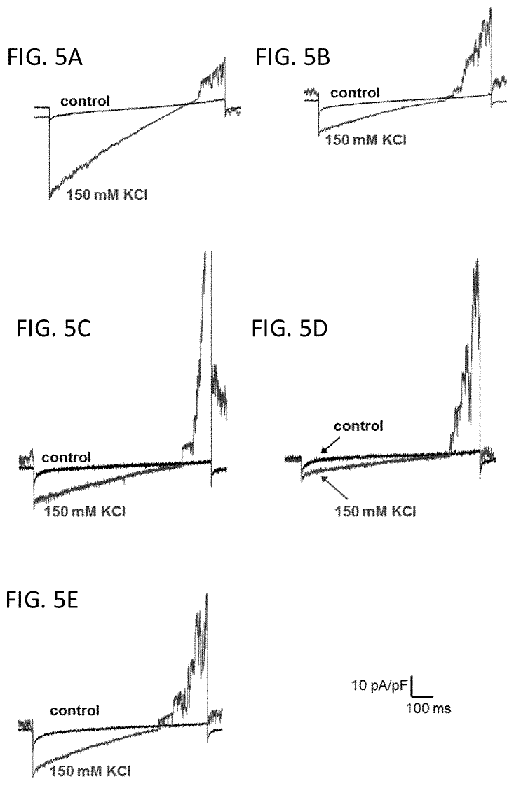

For all assays, the pipette solution contained 150 mM tetramethylammonium (TMA) hydroxide, 1.5 mM EGTA, 2 mM MgCl₂, and 150 mM HEPES, was tonicity adjusted to ˜370-450 mmol/kg with sucrose and pH adjusted to pH 7.2-7.3 with D Gluconic Acid, and the bath solution contained 150 mM KCl, 1 mM EGTA, and 40 mM HEPES, and was pH adjusted to pH 7.2 with TRIS base. FIGS. 5A-5E show exemplary recordings for heart, skeletal muscle, liver, kidney, and brown fat, respectively. The outward component is mediated by a chloride conductance, which is present when Cl⁻is included in the bath solution. Monovalent cation conductance could be seen for all tissues assayed.

E. Monovalent Cation Conductance in Mitoplasts from Different Species

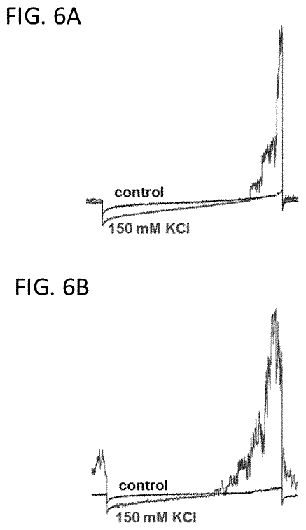

To determine if monovalent cation conductance is present other species, Drosophila and Caenorhabditis elegans mitoplasts were used as described in Example 1. For all assays, the pipette solution contained 110 mM Na-gluconate, 5 mM EGTA, 40 mM HEPES, 5 mM EDTA, and 1 mM MgCl₂ and was tonicity adjusted to ˜330 mmol/kg with sucrose and pH adjusted to pH 7 with NaOH, and the bath solution contained 150 mM KCl, 1 mM EGTA, and 40 mM HEPES, and was pH adjusted to pH 7 with TRIS base. FIGS. 6A-6B show exemplary recordings for Drosophila and C. elegans, respectively. Monovalent cation conductance could be seen for all species assayed.

F. The Effect of Heat on Monovalent Cation Conductance

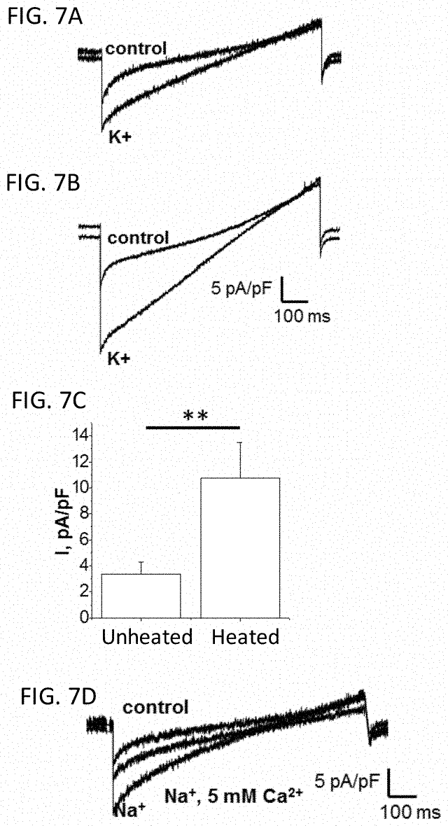

To determine if monovalent cation conductance was affected by heat, mitoplasts were used as described in Example 1 and were either not heated or were heated at 37° C. for 10-15 minutes in medium containing 750 mM KCl, 100 mM HEPES, and 1 mM EGTA (pH adjusted to 7.2 with KOH). For all assays, the pipette solution contained 125 mM K-gluconate, 1 mM EGTA, 40 mM HEPES, 1 mM EDTA, and 1 mM KCl and was tonicity adjusted to ˜350-370 mmol/kg with sucrose and pH adjusted to pH 7 with 1 M KOH, and the bath solution contained 125 mM K-gluconate, 1 mM EGTA, 40 mM HEPES, and 1 mM EDTA and was pH adjusted to pH 7 with 1 M KOH. FIGS. 7A-7B show exemplary recordings for not heated and heated samples, respectively. The current amplitudes measured at 5 mS after stepping the membrane from 0 to −160 mV and normalized to the total current are shown in FIG. 7C. Heating the mitoplast increased monovalent conductance significantly. The normalized conductance increase >3 fold when the mitoplast was heated.

G. The Effect of High Calcium on Monovalent Cation Conductance

To assess whether monovalent cation conductance in mitoplasts was altered by high concentrations of Ca²⁺, conductance was measured in the presence or absence of a high concentration of Ca²⁺.

For the assay, the pipette solution contained 110 mM Na-gluconate, 5 mM EGTA, 40 mM HEPES, 5 mM EDTA, and 1 mM NaCl and was tonicity adjusted to ˜350-370 mmol/kg with sucrose and pH adjusted to pH 7 with 1 M NaOH. The control bath solution contained 110 mM Na-gluconate, and 40 mM HEPES and was pH adjusted to pH 7 with 1 M NaOH. When assaying for the effect Ca²⁺, 5 mM CaCl₂) was added to the described bath solution. FIG. 7D show exemplary recordings in the presence and absence of a high concentration of Ca²⁺. Monovalent conductance was inhibited in the presence of a high concentration of Ca²⁺.

### Example 3: Inhibition of Monovalent Cation Conductance

To determine what known compounds inhibited the monovalent cation conductance, a large number of compounds were tested for their ability to inhibit monovalent cation conductance. The mitoplast patch-clamp was used as described in Example 1. For all assays, monovalent cation conductance was first measured with the described bath solution that did not comprise the inhibitor, and then for the same bath solution with the inhibitor, unless otherwise specified.

A. ATP Synthase and Adenine Nucleotide Translocator Inhibitors

Known ATP synthase inhibitors dicyclohexylcarbodiimide (DCCD) and oligomycin and ANT inhibitor carboxyatractyloside (CATR) were assessed for inhibitory effects on monovalent cation conductance.

For the DCCD assay, mitoplasts were prepared from wild-type C56BL/6 mice. The pipette solution contained 150 mM tetramethylammonium (TMA) hydroxide, 1.5 mM EGTA, 2 mM MgCl₂, and 150 mM HEPES, was tonicity adjusted to ˜450 mmol/kg with sucrose and pH adjusted to pH 7 with D Gluconic Acid, and the bath solution contained 150 mM KCl, 1.5 mM EGTA, and 40 mM HEPES, and was pH adjusted to pH 7.2 with 1 M KOH. When assaying for DCCD effect, 10 μM DCCD was added to the described bath solution. In addition,

For the oligomycin and CATR assays, the pipette solution contained 125 mM K-gluconate, 1 mM EGTA, 40 mM HEPES, 1 mM EDTA, and 1 mM KCl and was tonicity adjusted to ˜350-370 mmol/kg with sucrose and pH adjusted to pH 7 with 1 M KOH, and the bath solution contained 125 mM K-gluconate, 1 mM EGTA, 40 mM HEPES, and 1 mM EDTA and was pH adjusted to pH 7 with 1 M KOH. When assaying for inhibitor effect, 10 oligomycin or 5 μM CATR was added to the described bath solution.

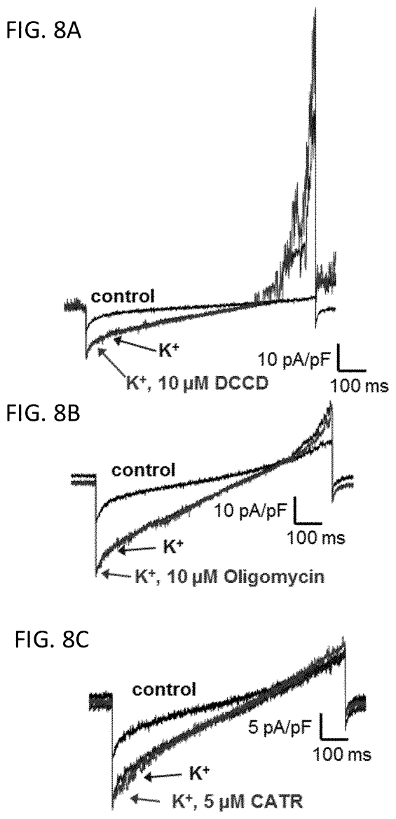

FIGS. 8A-8C show exemplary recordings for DCCD, oligomycin, and CATR treated mitoplasts, respectively. These known ATP synthase or ANT inhibitors had little to no effect on monovalent cation conductance.

B. K_{ATP} and ROMK Inhibitors

Known K_{ATP} inhibitors arachidonic acid (AA) and benzamil or ROMK inhibitor Tertiapin Q (TQ) were assessed for inhibitory effects on monovalent cation conductance.

For the K_{ATP} or ROMK inhibitor assays, the pipette solution contained 125 mM K-gluconate, 5 mM EGTA, 40 mM HEPES, 5 mM EDTA, and 1 mM KCl and was tonicity adjusted to ˜350-370 mmol/kg with sucrose and was pH adjusted to pH 7, or pH 8 for the benzamil assay, and the bath solution contained 125 mM K-gluconate, 1 mM EGTA, 40 mM HEPES, and 1 mM EDTA and was pH adjusted to pH 7, or pH 8 for the benzamil assay. When assaying for inhibitor effect, 1.5 μM AA, 0.5 μM TQ, or 10 μM benzamil was added to the described bath solution.

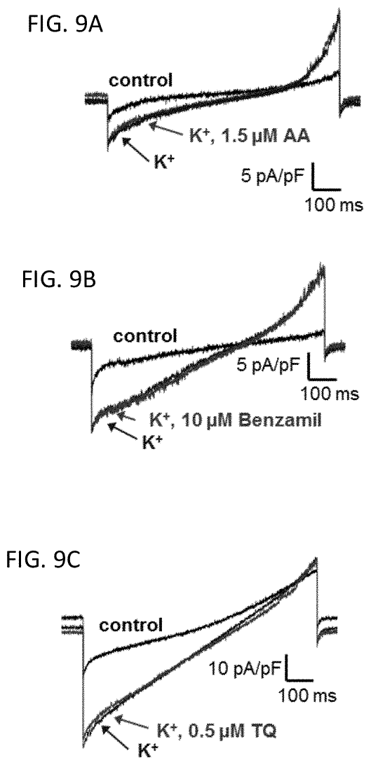

FIGS. 9A-9C show exemplary recordings for AA, benzamil, and TQ treated mitoplasts, respectively. These known K_{ATP} or ROMK inhibitors had little to no effect on monovalent cation conductance.

C. Other Monovalent Conductance Inhibitors

The amiloride derivative 5-(N-ethyl-N-isopropyl)amiloride (EIPA) was primarily considered an inhibitor of Na⁺ flux and the quinoline alkaloid quinine was primarily considered an inhibitor of K⁺ flux. EIPA and quinine were assayed using both Na⁺ and K⁺.

For the Na⁺ assays, the pipette solution contained 110 mM Na-gluconate, 5 mM EGTA, 40 mM HEPES, 5 mM EDTA, and 1 mM NaCl and was tonicity adjusted to ˜350-370 mmol/kg with sucrose and pH adjusted to pH 7 with 1 M NaOH. The bath solution contained 110 mM Na-gluconate, 5 mM EGTA, 40 mM HEPES, and 5 mM EDTA and was pH adjusted to pH 7 with 1 M NaOH. When assaying for inhibitor effect, 100 μM EIPA or 150 μM quinine was added to the described bath solution.

For the K⁺ assays, the pipette solution contained 110 mM K-gluconate, 1 or 5 mM EGTA, 40 mM HEPES, 1 or 5 mM EDTA, and 1 mM KCl and was tonicity adjusted to ˜350-370 mmol/kg with sucrose and pH adjusted to pH 7 with 1 M KOH, and the bath solution contained 110 mM K-gluconate, 1 or 5 mM EGTA, 40 mM HEPES, and 1 or 5 mM EDTA and was pH adjusted to pH 7 with 1 M KOH. When assaying for inhibitor effect, 80 μM EIPA or 150 μM quinine was added to the described bath solution.

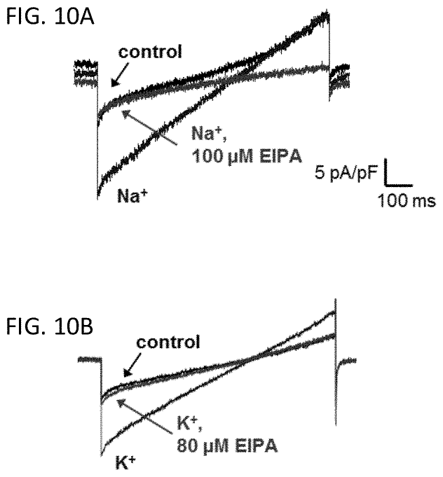

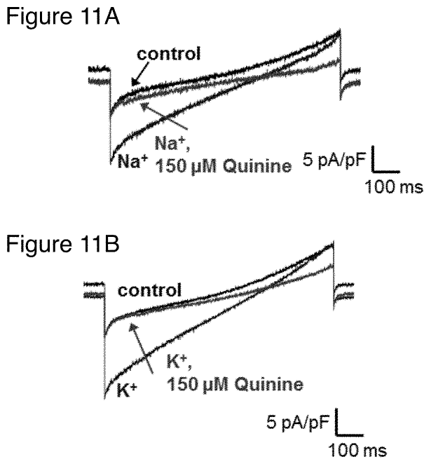

The amiloride derivative EIPA and the quinoline alkaloid quinine both significantly and almost completely inhibited monovalent cation conductance when either Na⁺ or K⁺ was used (FIGS. 10A-10B and 11A-11B). EIPA was primarily considered an inhibitor of Na⁺ flux and quinine was primarily considered an inhibitor of K⁺ flux.

D. Complex IV Inhibitors

Known Complex IV inhibitor potassium cyanide (KCN) was assessed for inhibitory effects on monovalent cation conductance.

For the KCN assay, mitoplasts were prepared from wild-type C56BL/6 mice. The pipette solution contained 150 mM tetramethylammonium hydroxide, 1.5 mM EGTA, 2 mM MgCl₂, and 150 mM HEPES, was tonicity adjusted to ˜450 mmol/kg with sucrose and pH adjusted to pH 7 with D Gluconic Acid, and the bath solution contained 150 mM KCl, 1.5 mM EGTA, and 40 mM HEPES and was pH adjusted to pH 7 with 1 M KOH. When assaying for KCN effect, 10 μM KCN was added to the described bath solution.

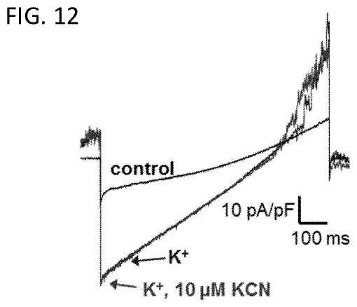

FIG. 12 shows exemplary recordings for KCN. This known Complex IV inhibitor had little to no effect on monovalent cation conductance.

E. Complex III Inhibitors

Known Complex III inhibitors antimycin, stigmatellin, and myxothiazol were assessed for inhibitory effects on monovalent cation conductance.

For the Complex III inhibitor assays, the pipette solution contained 125 mM K-gluconate, 1 mM EGTA, 40 mM HEPES, 1 mM EDTA, and 1 mM KCl and was tonicity adjusted to ˜350-370 mmol/kg with sucrose and was pH adjusted to pH 7 with 1 M KOH, and the bath solution contained 125 mM K-gluconate, 1 mM EGTA, 40 mM HEPES, and 1 mM EDTA and was pH adjusted to pH 7 with 1 M KOH. When assaying for inhibitor effect, 10 μM antimycin, 0.5 μM stigmatellin, or 10 μM myxothiazol was added to the described bath solution.

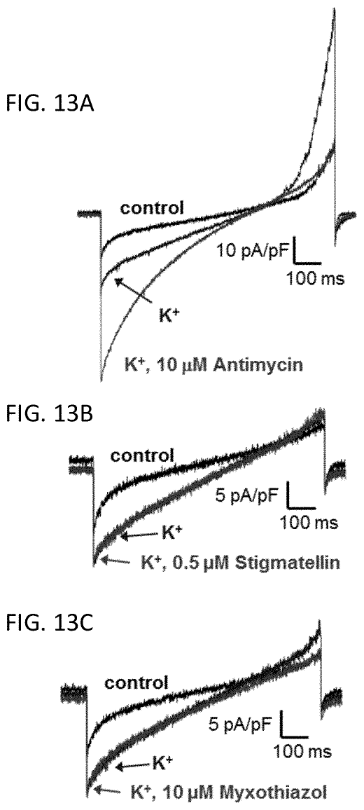

FIGS. 13A-13C show exemplary recordings for antimycin, stigmatellin, and myxothiazol treated mitoplasts, respectively. These known Complex III inhibitors had little to no effect on monovalent cation conductance. Antimycin at the concentration used elicited an uncoupling H⁺ current that overlaps with the K⁺ current.

F. Complex II Inhibitors

Known Complex II inhibitor atpenin was assessed for inhibitory effects on monovalent cation conductance. It has also been previously suggested that atpenin can affect a K_{ATP} channel of the mitochondria.

For the Complex II inhibitor assay, the pipette solution contained 125 mM K-gluconate, 1 mM EGTA, 40 mM HEPES, 5 mM EDTA, and 1 mM KCl and was tonicity adjusted to ˜350-370 mmol/kg with sucrose and pH adjusted to pH 7 with 1 M KOH, and the bath solution contained 125 mM K-gluconate, 1 mM EGTA, 40 mM HEPES, and 1 mM EDTA and was pH adjusted to pH 7 with 1 M KOH. When assaying for atpenin effect, 0.2 μM or 1 μM atpenin was added to the described bath solution.

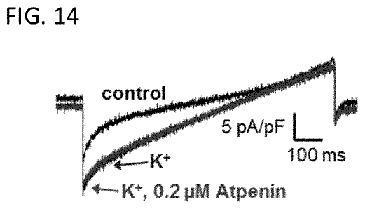

FIG. 14 shows exemplary recordings for 0.2 μM atpenin treated mitoplasts. This known Complex II inhibitor and proposed K_{ATP} inhibitor had little to no effect on monovalent cation conductance.

G. Other Electron Transport Chain Inhibitors

Known or suspected electron transport chain inhibitors rotenone, fenazaquin, piericidin, 1-methyl-4-phenylpyridinium (MPP+), bullatacin, fenipiroximate, metformin, proguanil, and phenformin were assessed for inhibitory effects on monovalent cation conductance.

1. Methods

a. 110 mM Na⁺ Conditions

For the rotenone and fenazaquin assays, the pipette solution contained 110 mM Na-gluconate, 5 mM EGTA, 40 mM HEPES, 5 mM EDTA, and 1 mM NaCl and was tonicity adjusted to ˜350-370 mmol/kg with sucrose and pH adjusted to pH 7 with 1 M NaOH. The bath solution contained 110 mM Na-gluconate, 5 mM EGTA, 40 mM HEPES, and 5 mM EDTA and was pH adjusted to pH 7 with 1 M NaOH. When assaying for inhibitor effect, 10 μM rotenone or 10 μM fenazaquin was added to the described bath solution.

b. 110 mM K⁺ and 125 mM K⁺ Conditions

For the piericidin, MPP+, bullatacin, rolliniastatin, and fenipiroximate assays, the pipette solution contained 110 mM (piericidin) or 125 mM (MPP+, bullatacin, rolliniastatin, and fenipiroximate) K-gluconate, 1 or 5 mM EGTA, 40 mM HEPES, 1 or 5 mM EDTA, and 1 mM KCl and was tonicity adjusted to ˜350-370 mmol/kg with sucrose and pH adjusted to pH 7 with 1 M KOH, and the bath solution contained 110 mM (piericidin) or 125 mM (MPP+, bullatacin, rolliniastatin, and fenipiroximate) K-gluconate, 1 or 5 mM EGTA, 40 mM HEPES, and 1 or 5 mM EDTA and was pH adjusted to pH 7 with 1 M KOH. When assaying for inhibitor effect, 1 μM piericidin, 0.5 mM MPP+, 1 μM bullatacin, 2 μM rolliniastatin, or 0.5 μM fenipiroximate was added to the described bath solution.

c. Biguanides Assay

For the Metformin HCl, phenformin, and proguanil assays, the pipette solution contained 125 mM K-gluconate, 1 mM EGTA, 40 mM HEPES, 1 mM EDTA, and 1 mM KCl and was tonicity adjusted to ˜350-370 mmol/kg with sucrose and pH adjusted to pH 7, with 1 M KOH, and the bath solution contained 125 mM K-gluconate, 1 mM EGTA, 40 mM HEPES, and 1 mM EDTA and was pH adjusted to pH 7, with 1 M KOH. When assaying for phenformin or proguanil effect, 1 mM phenformin or 100 μM proguanil was added to the described bath solution. When assaying for metformin effect, the described bath was replaced with a metformin solution comprising 30 mM metformin HCl, 1 mM EGTA, 1 mM EDTA, and 40 mM HEPES, which was pH adjusted to pH 7 using TRIS base. This modification of the protocol was used to maintain the osmolarity of the bath solution when using 30 mM metformin HCl.

2. Results

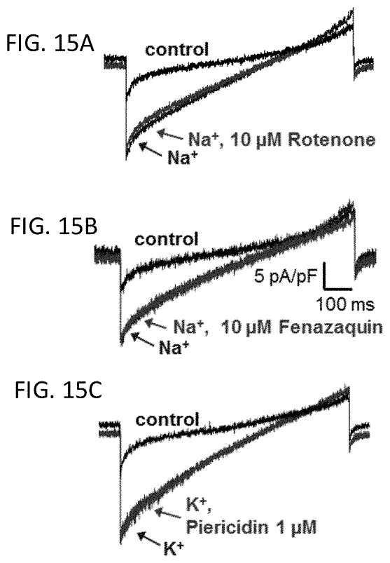

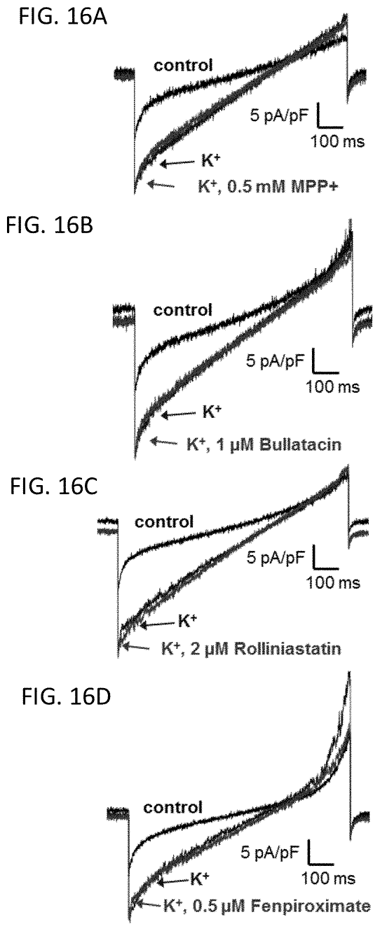

Rotenone, fenazaquin, or piericidin (FIGS. 15A-15C, respectively) and MPP+, bullatacin, rolliniastatin, or fenipiroximate (FIGS. 16A-16D, respectively) had little to no effect on monovalent cation conductance.

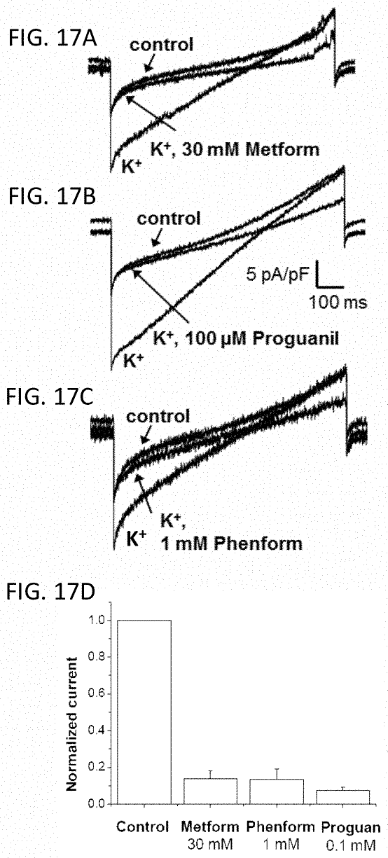

The biguanides metformin, proguanil, and phenformin also almost completely inhibited monovalent cation conductance (FIG. 17A-17C). The current amplitudes measured at 5 mS after stepping the membrane from 0 to −160 mV and normalized to the total current are shown in FIG. 17D.

H. Potent Inhibitors of Monovalent Cation Conductance

To further assess whether other amiloride derivatives inhibit monovalent cation conductance, amiloride derivatives AA6, 5o, and 5m were assayed.

For the assays, the pipette solution contained 125 mM K-gluconate, 1 mM EGTA, 40 mM HEPES, 1 mM EDTA, and 1 mM KCl and was tonicity adjusted to ˜350-370 mmol/kg with sucrose and pH adjusted to pH 7 with 1 M KOH, and the bath solution contained 125 mM K-gluconate, 1 mM EGTA, 40 mM HEPES, and 1 mM EDTA and was pH adjusted to pH 7, or pH 8 for the benzamil assay, with 1 M KOH. When assaying for inhibitor effect, 200 nM AA6, 1 μM 5o, or 1 μM 5m was added to the described bath solution.

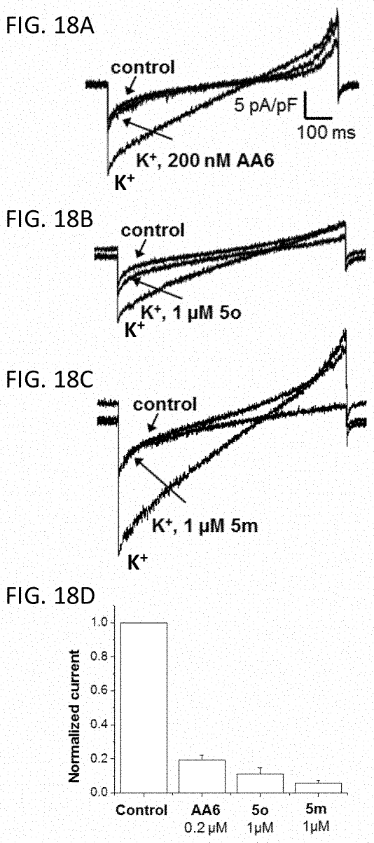

The amiloride derivatives AA6, 5o, and 5m almost completely inhibited monovalent cation conductance at concentrations far below what was used for the other inhibitors tested (FIG. 18A-18C). The current amplitudes measured at 5 mS after stepping the membrane from 0 to −160 mV and normalized to the total current are shown in FIG. 18D.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-99. (canceled)

100. A method of measuring the inner mitochondrial membrane nonselective monovalent cation conductance of a mitochondrion, comprising the steps of:
treating the mitochondrion to make the inner mitochondrial membrane thereof accessible, wherein the treated mitochondrion is in a bath solution, wherein a reference electrode is present in the bath solution;
contacting a patch clamp recording electrode to the inner mitochondrial membrane and forming a seal therewith, wherein the patch clamp electrode comprises a matrix solution;
applying voltage to induce the flow of monovalent cations across the inner mitochondrial membrane; and
obtaining measurements that indicate the magnitude of monovalent cation current across the inner mitochondrial membrane; and
wherein the mitochondrial calcium uniporter activity in the inner mitochondrial membrane is inhibited.

101. The method of claim 100, wherein
the inner mitochondrial membrane nonselective monovalent cation conductance is a conductance that can be inhibited by metformin.

102. The method of claim 100, wherein
the treatment to make the inner mitochondrial membrane accessible is the formation of a mitoplast.

103. The method of claim 100, wherein
the bath solution comprises
a monovalent cation;
a chelator which removes substantially all divalent cations from the solution;
a buffer;
a sugar or sugar alcohol;
wherein the bath solution does not comprise chloride;
wherein the pH of the bath solution is between about 6.5 and 9; and
wherein the osmolality of the bath solution is between about 250 to about 500 mmol/kg.

104. The method of claim 103, wherein
the bath solution comprises a monovalent cation comprising Na, K, Li, Cs, TMA, or a mixture of the foregoing.

105. The method of claim 103, wherein
the monovalent cation is provided to the bath solution in the form of a gluconate salt.

106. The method of claim 103, wherein
the chelator of the bath solution is a mixture of EDTA and EGTA.

107. The method of claim 100, wherein
the matrix solution comprises
a monovalent cation;
a chelator which removes substantially all divalent cations from the solution;
a buffer;
a sugar or sugar alcohol;
wherein the matrix solution does not comprise chloride;
wherein the pH of the bath solution is between about 6.5 and 9; and
wherein the osmolality of the bath solution is between about 250 to about 500 mmol/kg.

108. The method of claim 107, wherein
the matrix solution comprises a monovalent cation comprising Na, K, Li, Cs, TMA, or a mixture of the foregoing.

109. The method of claim 107, wherein
the monovalent cation is provided to the matrix solution in the form of a gluconate salt.

110. The method of claim 107, wherein
the chelator of the matrix solution is a mixture of EDTA and EGTA.

111. The method of claim 100, wherein
the mitochondrial calcium uniporter conductance of the IMM is inhibited by
the use of measurement conditions wherein calcium is absent;
the application of a mitochondrial calcium uniporter inhibitor; or
by the use of mitochondria comprising mutations that reduce the activity of the mitochondrial calcium uniporter.

112. The method of claim 100, wherein
the mitochondrion is heated prior to and/or during the measurement process by use of a bath solution having a temperature between 30 and 37° C.

113. The method of claim 100, wherein
the mitochondrion is cooled by placement of the bath solution on ice for a period of 15-30 minutes prior to performing the measurement.

114. The method of claim 100, wherein
the bath solution and/or matrix solution comprises an inhibitor or putative inhibitor of the inner mitochondrial membrane nonselective monovalent cation conductance.

115. A method of identifying an inhibitor of the inner mitochondrial membrane nonselective monovalent cation conductance, comprising
treating a mitochondrion to make the inner mitochondrial membrane thereof accessible, wherein the treated mitochondrion is in a bath solution, and wherein a reference electrode is present in the bath solution;
contacting the inner mitochondrial membrane with a test compound comprising a putative inhibitor of the inner mitochondrial membrane nonselective monovalent cation conductance;
and
measuring an inner mitochondrial membrane monovalent cation conductance by the steps of:
contacting a patch clamp recording electrode to the inner mitochondrial membrane and forming a seal therewith, wherein the patch clamp electrode comprises a matrix solution;
applying voltage to induce the flow of monovalent cations across the inner mitochondrial membrane;
obtaining measurements that indicate the magnitude of monovalent cation current across the inner mitochondrial membrane; and
wherein the mitochondrial calcium uniporter activity in the inner mitochondrial membrane is inhibited;
and
wherein, if the magnitude of monovalent cation current across the inner mitochondrial membrane is reduced by a selected threshold level of inhibition compared to control treatments lacking the putative inhibitor of the inner mitochondrial membrane nonselective monovalent cation conductance, the test compound is deemed to be an inhibitor of the inner mitochondrial membrane nonselective monovalent cation conductance.

116. A composition comprising 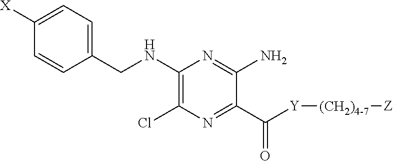
or a tautomer thereof, or a salt of any of the foregoing, wherein:
X is halo;
Y is 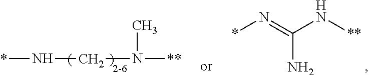
wherein:
* indicates the point of attachment to the carbonyl of the parent structure and ** indicates the point of attachment to (CH₂)₄₋₇—Y; and
Z is C₁-C₆alkyl or phenyl.

117. The composition of claim 116, comprising

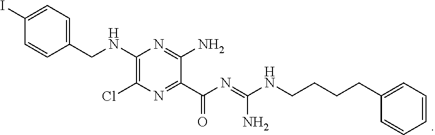 [SMILES: N/C(=N\C(=O)c1nc(Cl)c(NCc2ccc(I)cc2)nc1N)NCCCCc1ccccc1]

118. The composition of claim 116, comprising

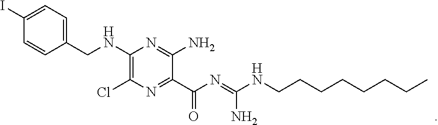 [SMILES: CCCCCCCCN/C(N)=N/C(=O)c1nc(Cl)c(NCc2ccc(I)cc2)nc1N]

119. The composition of claim 116, comprising

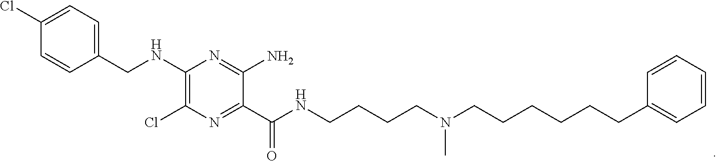 [SMILES: CN(CCCCCCc1ccccc1)CCCCNC(=O)c1nc(Cl)c(NCc2ccc(Cl)cc2)nc1N]

120. The composition of claim 116, wherein
the composition comprises any of:
a quinoline alkaloid;
a biguanide; and
5-(N-ethyl-N-isopropyl)amiloride.

121. A method of treating a disease or condition wherein the disease or condition is associated with dysregulated mitochondrial function, comprising
administering to the subject an effective amount of an inhibitor of the mitochondrial inner membrane nonspecific monovalent cation conductance, wherein the inhibitor comprises 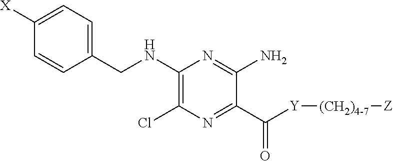
or a tautomer thereof, or a salt of any of the foregoing, wherein:
X is halo;
Y is 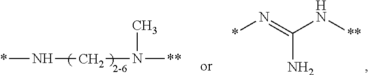
wherein:
* indicates the point of attachment to the carbonyl of the parent structure and ** indicates the point of attachment to (CH₂)₄₋₇—Y; and
Z is C₁-C₆ alkyl or phenyl.

122. The method of claim 121, wherein the inhibitor comprises:

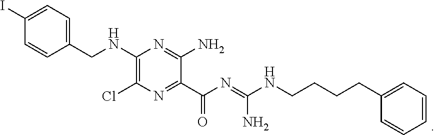 [SMILES: N/C(=N\C(=O)c1nc(Cl)c(NCc2ccc(I)cc2)nc1N)NCCCCc1ccccc1]

123. The method of claim 121, wherein the inhibitor comprises:

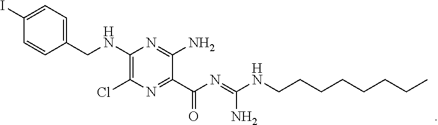 [SMILES: CCCCCCCCN/C(N)=N/C(=O)c1nc(Cl)c(NCc2ccc(I)cc2)nc1N]

124. The method of claim 121, wherein the inhibitor comprises:

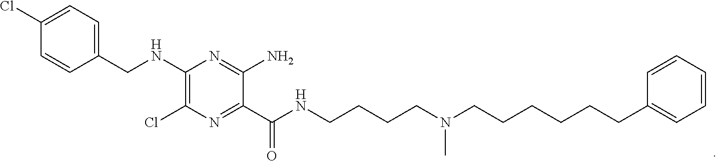 [SMILES: CN(CCCCCCc1ccccc1)CCCCNC(=O)c1nc(Cl)c(NCc2ccc(Cl)cc2)nc1N]

125. The method of claim 121, wherein
the condition is selected from the group consisting of diabetes mellitus, Type 1 diabetes, Type 2 diabetes, prediabetes, gestational diabetes, autoimmune destruction or impairment of pancreatic islet beta cells, reduced insulin production, insulin resistance, dysregulated glucose metabolism, progression from non-overt diabetic status to overt diabetic status, elevated fasting blood glucose concentration hyperglycemia, and diabetic ketoacidosis.

126. The method of claim 121, wherein
the condition is cancer.

127. The method of claim 121, wherein
the condition is a cardiac or ischemic condition.

128. The method of claim 121, wherein
the cardiac or ischemic condition is selected from the group consisting of vascular occlusion or constriction, insufficient blood circulation, ischemia or stroke, mini-stroke, or micro-infarct, coronary artery disease, heart attack, myocardial infarction, carotid artery disease, peripheral arterial disease, critical limb ischemia, claudication, cerebrovascular disease, reduced circulation in the brain, arterial occlusive disease, hypoperfusion, atherosclerosis, thrombosis, and embolism.
